(19) United States
(12) Patent Application Publication
(10) Pub. No.: US 2009/0018148 A1
(43) Pub. Date: Jan. 15, 2009

(54) Xanthine Derivatives, Processes For Preparing Them And Their Uses

(75) Inventors: Florence Moureau, Wauthier-Braine (BE); Benoit Kenda, Emines (BE); Frank Driessens, Brussels (BE); Luc Quere, Sombreffe (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Brussels (BE)

(21) Appl. No.: 12/096,340

(22) PCT Filed: Nov. 30, 2006
(86) PCT No.: PCT/EP2006/011501

§ 371 Date: Sep. 18, 2008

(30) Foreign Application Priority Data
Dec. 7, 2005 (EP) 05026692.3

(51) Int. Cl. A61K 31/522; C07D 473/04; A61P 25/00

(52) U.S. Cl. 514/263.33; 544/266

(57) ABSTRACT

The present invention concerns xanthine derivatives, having formula (I), processes for preparing them, pharmaccutical compositions containing them and their use as pharmaceuticals.

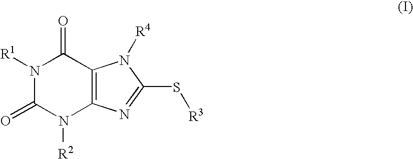

The present invention concerns xanthine derivatives, having formula (I), processes for preparing them, pharmaceutical compositions containing them and their use as pharmaceuticals.

The present invention concerns xanthine derivatives, processes for preparing them, pharmaceutical compositions containing them and their use as pharmaceuticals.

The present invention concerns xanthine derivatives, having formula (I), processes for preparing them, pharmaceutical compositions containing them and their use as pharmaceuticals.

The present invention concerns xanthine derivatives, processes for preparing them, pharmaceutical compositions containing them and their use as pharmaceuticals.

Some xanthine derivatives interacting with adenosine A1 and A2 receptors have been shown to be either proconvulsant or anticonvulsant depending upon the seizure model use (Klitgaard et al. Eur. J. Pharmacol. 1993, 242, (3) 221-8).

It has been found that certain xanthine derivatives demonstrate markedly improved therapeutic properties and shows in vitro affinities for Levetiracetam Binding Site (LBS)/SV2 protein.

In one aspect the invention therefore provides compounds having formula I, their enantiomers, diastereoisomers and mixtures thereof (including all possible mixtures of stereoisomers), or pharmaceutically acceptable salts thereof,

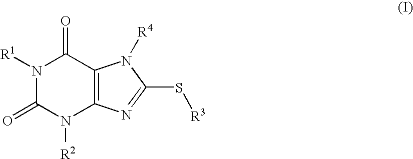

wherein

R¹ is hydrogen or C₁₋₆ alkyl;

R² is hydrogen or C₁₋₄ alkyl;

R³ is a group of formula —CHR⁵R⁶ or a benzyl group;

R⁴ is C₁₋₈ alkyl optionally substituted by alkoxycarbonyl, C₃₋₆ cycloalkyl, aryl or heterocycle;

R⁵ is C₂₋₄ alkyl;

R⁶ is C₂₋₄ alkyl, amido or —COOR⁷;

R⁷ is C₁₋₄ alkyl;

Without prejudice to their novel therapeutic use the following compounds are excluded from the product claims:
When R¹ is hydrogen, R² is methyl, R³ is —CHR⁵R⁶, R⁶ is ethoxycarbonyl and R⁵ is ethyl, then R⁴ is different from methyl, n-propyl, i-propyl, n-pentyl, n-heptyl, 3-bromobenzyl, 4-chlorobenzyl, 4-methylbenzyl or 2-phenylethyl;
When R¹ is hydrogen, R² is methyl, R³ is benzyl, then R⁴ is different from i-propyl, n-butyl, 3-methylbutyl, benzyl, phenylethyl-, or 3-phenylpropyl;
When R¹ and R² are methyl, R³ is benzyl, R⁴ is different from methyl, 3-methylbutyl, benzyl, 3-phenylpropyl or 4-chlorophenylmethyl;
Finally 8-(2-chloro-benzylsulfanyl)-3-methyl-7-octyl-3,7-dihydro-purine-2,6-dione is excluded.

Usually when R³ is a benzyl group, then R⁴ is C₁₋₈ alkyl optionally substituted by alkoxycarbonyl.

Usually when R³ is a group of formula —CHR⁵R⁶, then R⁴ is C₁₋₈ alkyl optionally substituted by C₃₋₆ cycloalkyl, aryl or heterocycle.

The term “alkyl”, as used herein, is a group which represents saturated, monovalent hydrocarbon radicals having straight (unbranched) or branched moieties, or combinations thereof, and containing 1-8 carbon atoms, preferably 1-6 carbon atoms; more preferably alkyl groups have 1-4 carbon atoms. Alkyl moieties may optionally be substituted by 1 to 5 substituents independently selected from the group consisting of hydroxy, alkoxy, cyano, ethynyl, alkoxycarbonyl, acyl, aryl or heterocycle. Alkyl moieties may be optionally substituted by a cycloalkyl as defined hereafter. Preferred alkyl groups according to the present invention are methyl, cyanomethyl, ethyl, 2-ethoxy-2-oxoethyl, 2-methoxyethyl, n-propyl, 2-oxopropyl, 3-hydroxypropyl, 2-propynyl, n-butyl, i-butyl, n-pentyl, 3-pentyl, n-hexyl, cyclohexylmethyl, benzyl, 2-bromobenzyl, 3-bromobenzyl, 4-bromobenzyl, 3-methoxybenzyl, 3-nitrobenzyl, 3-aminobenzyl, 4-(aminosulfonyl)benzyl, 1-phenylethyl, 2-phenylethyl, (3,5-dimethylisoxazol-4-yl)methyl or (5-nitro-2-furyl)methyl. More preferred alkyl groups are methyl, ethyl, cyanomethyl, 2-methoxyethyl, n-propyl, 3-hydroxypropyl, 2-propynyl, n-butyl, 3-pentyl, n-hexyl, benzyl, 3-bromobenzyl, 3-methoxybenzyl, 3-nitrobenzyl, 3-aminobenzyl, (3,5-dimethylisoxazol-4-yl)methyl or (5-nitro-2-furyl)methyl. Most preferred alkyl groups are methyl, ethyl, 3-methoxybenzyl, 3-nitrobenzyl or (5-nitro-2-furyl)methyl.

The term “cycloalkyl”, as used herein, represents a monovalent group of 3 to 8, preferably 3 to 6 carbon atoms derived from a saturated cyclic hydrocarbon, which may be substituted by any suitable group including but not limited to one or more moieties selected from groups as described above for the alkyl groups. Preferred cycloalkyl group according to the present invention is cyclohexyl.

The term “aryl” as used herein, is defined as a phenyl group optionally substituted by 1 to 4 substituents independently selected from halogen, amino, nitro, alkoxy or aminosulfonyl. Preferred aryl groups are phenyl, 2-bromophenyl, 3-bromophenyl, 4-bromophenyl, 3-methoxyphenyl, 3-nitrophenyl, 3-aminophenyl or 4-(aminosulfonyl)phenyl.

The term “phenyl”, as used herein, represents an aromatic hydrocarbon group of formula —C₆H₅.

The term “benzyl group”, as used herein, represents a group of formula —CH₂-aryl. Preferred benzyl groups are benzyl, 2-bromobenzyl, 3-bromobenzyl, 4-bromobenzyl, 3-methoxybenzyl, 3-nitrobenzyl, 3-aminobenzyl or 4-(aminosulfonyl)benzyl. More preferred benzyl groups are benzyl, 3-bromobenzyl, 3-methoxybenzyl, 3-nitrobenzyl or 3-aminobenzyl. Most preferred alkyl groups are 3-methoxybenzyl or 3-nitrobenzyl.

The term “halogen”, as used herein, represents an atom of fluorine, chlorine, bromine, or iodine. Preferred halogen is bromine.

The term “hydroxy”, as used herein, represents a group of formula —OH.

The term “cyano”, as used herein, represents a group of formula —CN.

The term “amino”, as used herein, represents a group of formula —NH₂.

The term “ethynyl”, as used herein, represents a group of formula —C≡CH.

The term “alkoxy”, as used herein, represents a group of formula —OR^{a} wherein R^{a} is an alkyl group, as defined above. Preferred alkoxy group is methoxy.

The term “nitro”, as used herein, represents a group of formula —NO₂.

The term “amido”, as used herein, represents a group of formula —C(═O)NH₂.

The term “acyl”, as used herein, represents a group of formula —C(═O)R^{b} wherein R^{b} is an alkyl group, as defined here above. Preferred acyl group is acetyl (—C(═O)Me).

The term “alkoxycarbonyl (or ester)”, as used herein, represents a group of formula —COOR^{c} wherein R^{c} is an alkyl group; with the proviso that R^{c} does not represent an alkyl alpha-substituted by hydroxy. Preferred alkoxycarbonyl group is ethoxycarbonyl.

The term “heterocycle”, as used herein, represents a 5-membered ring containing one or two heteroatoms selected from O or N. The heterocycle may be substituted by one or two C₁₋₄ alkyl or nitro. Preferred heterocycles are (3,5-dimethylisoxazol-4-yl) or (5-nitro-2-furyl). Most preferred heterocycle is (5-nitro-2-furyl).

Generally R¹ is hydrogen or C₁₋₆ alkyl. Usually R¹ is hydrogen or C₁₋₆ alkyl optionally substituted by hydroxy, alkoxy, cyano, ethynyl, alkoxycarbonyl or acyl. Preferably R¹ is hydrogen, methyl, cyanomethyl, 2-ethoxy-2-oxoethyl, 2-methoxyethyl, n-propyl, 2-oxopropyl, 3-hydroxypropyl, 2-propynyl, n-pentyl or n-hexyl. More preferably R¹ is hydrogen, methyl, cyanomethyl, 2-methoxyethyl, n-propyl, 3-hydroxypropyl or 2-propynyl. Most preferably R¹ is hydrogen.

Generally R² is hydrogen or C₁₋₄ alkyl. Usually R² is hydrogen or unsubstituted C₁₋₄ alkyl. Preferably R² is hydrogen, methyl or n-butyl. More preferably, R² is methyl.

Generally R³ is a group of formula —CHR⁵R⁶ or a benzyl group. Preferably R³ is 3-pentyl, 1-(aminocarbonyl)propyl, 1-(ethoxycarbonyl)propyl or 3-bromobenzyl. Most preferably R³ is 1-(ethoxycarbonyl)propyl.

Generally R⁴ is C₁₋₈ alkyl optionally substituted by alkoxycarbonyl, C₃₋₆ cycloalkyl, aryl or heterocycle. Usually R⁴ is C₁₋₈ alkyl optionally substituted by cyclohexyl, phenyl, bromophenyl, aminophenyl, methoxyphenyl, nitrophenyl, aminosulfonylphenyl, 3,5-dimethylisoxazol-4-yl, 5-nitro-2-furyl or ethoxycarbonyl. Preferably R⁴ is n-butyl, i-butyl, n-pentyl, n-hexyl, cyclohexylmethyl, benzyl, 2-bromobenzyl, 3-bromobenzyl, 4-bromobenzyl, 3-methoxybenzyl, 3-nitrobenzyl, 3-aminobenzyl, 4-(aminosulfonyl)benzyl, 1-phenylethyl, 2-phenylethyl, (3,5-dimethylisoxazol-4-yl)methyl, (5-nitro-2-furyl)methyl or 1-(ethoxycarbonyl)propyl. More preferably R⁴ is n-butyl, n-hexyl, benzyl, 3-bromobenzyl, 3-methoxybenzyl, 3-nitrobenzyl, 3-aminobenzyl, (3,5-dimethylisoxazol-4-yl)methyl, (5-nitro-2-furyl)methyl or 1-(ethoxycarbonyl)propyl. Most preferably R⁴ is 3-methoxybenzyl, 3-nitrobenzyl or (5-nitro-2-furyl)methyl.

Generally R⁵ is C₂₋₄ alkyl. Usually R⁵ is unsubstituted C₂₋₄ alkyl. Preferably R⁵ is ethyl.

Generally R⁶ is C₂₋₄ alkyl, amido or —COOR⁷. Usually R⁶ is unsubstituted C₂₋₄ alkyl, amido or —COOR⁷. Preferably R⁶ is ethyl, amido or ethoxycarbonyl. Most preferably R⁶ is ethoxycarbonyl.

Generally R⁷ is C₁₋₄ alkyl. Usually R⁷ is unsubstituted C₁₋₄ alkyl. Preferably, R⁷ is ethyl.

Usually the invention provides compounds having formula I, their enantiomers, diastereoisomers and mixtures thereof (including all possible mixtures of stereoisomers), or pharmaceutically acceptable salts thereof,

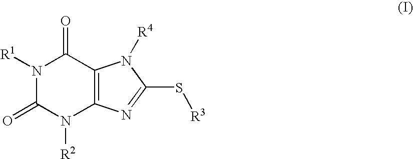

wherein

R¹ is hydrogen, C₁₋₆ alkyl optionally substituted by hydroxy, alkoxy, cyano, ethynyl, alkoxycarbonyl or acyl;

R² is hydrogen or unsubstituted C₁₋₄ alkyl;

R³ is a group of formula —CHR⁵R⁶ or a benzyl group;

R⁴ is C₁₋₈ alkyl optionally substituted by cyclohexyl, phenyl, bromophenyl, aminophenyl, methoxyphenyl, nitrophenyl, aminosulfonylphenyl, 3,5-dimethylisoxazol-4-yl, 5-nitro-2-furyl or ethoxycarbonyl;

R⁵ is unsubstituted C₂₋₄ alkyl;

R⁶ is unsubstituted C₂₋₄ alkyl, amido or —COOR⁷;

R⁷ is unsubstituted C₁₋₄ alkyl;

with the proviso that when R¹ is hydrogen, R² is methyl, R³ is —CHR⁵R⁶, R⁶ is ethoxycarbonyl and R⁵ is ethyl, then R⁴ is different from n-propyl, i-propyl, n-pentyl, n-heptyl, 3-bromobenzyl, 4-chlorobenzyl, 4-methylbenzyl or 2-phenylethyl.

In the above embodiment, preferably, when R³ is a benzyl group, then R⁴ is C₁₋₈ alkyl optionally substituted by alkoxycarbonyl.

In the above embodiment, preferably, when R³ is a group of formula —CHR⁵R⁶, then R⁴ is C₁₋₈ alkyl optionally substituted by C₃₋₆ cycloalkyl, aryl or heterocycle.

In a preferred embodiment,

R¹ is hydrogen, methyl, cyanomethyl, 2-ethoxy-2-oxoethyl, 2-methoxyethyl, n-propyl, 2-oxopropyl, 3-hydroxypropyl, 2-propynyl, n-pentyl or n-hexyl;

R² is hydrogen, methyl or n-butyl;

R³ is 3-pentyl, 1-(aminocarbonyl)propyl, 1-(ethoxycarbonyl)propyl or 3-bromobenzyl;

R⁴ is n-butyl, i-butyl, n-pentyl, n-hexyl, cyclohexylmethyl, benzyl, 2-bromobenzyl, 3-bromobenzyl, 4-bromobenzyl, 3-methoxybenzyl, 3-nitrobenzyl, 3-aminobenzyl, 4-(aminosulfonyl)benzyl, 1-phenylethyl, 2-phenylethyl, (3,5-dimethylisoxazol-4-yl)methyl, (5-nitro-2-furyl)methyl or 1-(ethoxycarbonyl)propyl;

with the proviso that when R¹ is hydrogen, R² is methyl and R³ is 1-(ethoxycarbonyl)propyl, then R⁴ is different from n-pentyl, 3-bromobenzyl or 2-phenylethyl.

In the above embodiment, preferably, when R³ is 3-bromobenzyl, then R⁴ is C₁₋₈ alkyl optionally substituted by alkoxycarbonyl.

In the above embodiment, preferably, when R³ is 3-pentyl, 1-(aminocarbonyl)propyl or 1-(ethoxycarbonyl)propyl, then R⁴ is different from 1-(ethoxycarbonyl)propyl.

In a more preferred embodiment, R¹ is hydrogen, methyl, cyanomethyl, 2-methoxyethyl, n-propyl, 3-hydroxypropyl or 2-propynyl;

R² is methyl;

R³ is 3-pentyl, 1-(aminocarbonyl)propyl, 1-(ethoxycarbonyl)propyl or 3-bromobenzyl;

R⁴ is n-butyl, n-hexyl, benzyl, 3-bromobenzyl, 3-methoxybenzyl, 3-nitrobenzyl, 3-aminobenzyl, (3,5-dimethylisoxazol-4-yl)methyl, (5-nitro-2-furyl)methyl or 1-(ethoxycarbonyl)propyl;

with the proviso that when R¹ is hydrogen, R² is methyl and R³ is 1-(ethoxycarbonyl)propyl, then R⁴ is different from 3-bromobenzyl.

In the above embodiment, preferably, when R³ is 3-bromobenzyl, then R⁴ is 1-(ethoxycarbonyl)propyl;

In the above embodiment, preferably, when R³ is 3-pentyl, 1-(aminocarbonyl)propyl or 1-(ethoxycarbonyl)propyl, then R⁴ is different from 1-(ethoxycarbonyl)propyl;

In a most preferred embodiment, R¹ is hydrogen; R² is methyl; R³ is 1-(ethoxycarbonyl)propyl; and R⁴ is 3-methoxybenzyl, 3-nitrobenzyl or (5-nitro-2-furyl)methyl.

A further embodiment consists in compounds wherein R² is methyl, R³ is a group of formula —CHR⁵R⁶ with R⁵ being C₂₋₄ alkyl, R⁶ being amido or —COOR⁷ and R⁷ being methyl or ethyl.

Preferred compounds are ethyl 2-[(7-benzyl-1,3-dimethyl-2,6-dioxo-2,3,6,7-tetrahydro-1H-purin-8-yl)thio]butanoate; ethyl 2-{[7-(3-bromobenzyl)-1-(2-ethoxy-2-oxoethyl)-3-methyl-2,6-dioxo-2,3,6,7-tetrahydro-1H-purin-8-yl]thio}butanoate; ethyl 2-{[7-(3-bromobenzyl)-1-(2-methoxyethyl)-3-methyl-2,6-dioxo-2,3,6,7-tetrahydro-1H-purin-8-yl]thio}butanoate; ethyl 2-{[7-(3-bromobenzyl)-2,6-dioxo-2,3,6,7-tetrahydro-1H-purin-8-yl]thio}butanoate; ethyl 2-{[7-(3-bromobenzyl)-1,3-dimethyl-2,6-dioxo-2,3,6,7-tetrahydro-1H-purin-8-yl]thio}butanoate; ethyl 2-{[7-(2-bromobenzyl)-1,3-dimethyl-2,6-dioxo-2,3,6,7-tetrahydro-1H-purin-8-yl]thio}butanoate; ethyl 2-{[7-(3-bromobenzyl)-1-(cyanomethyl)-3-methyl-2,6-dioxo-2,3,6,7-tetrahydro-1H-purin-8-yl]thio}butanoate; ethyl 2-{[7-(3-bromobenzyl)-3-methyl-2,6-dioxo-1-propyl-2,3,6,7-tetrahydro-1H-purin-8-yl]thio}butanoate; ethyl 2-{[7-(3-bromobenzyl)-3-methyl-2,6-dioxo-1-(2-oxopropyl)-2,3,6,7-tetrahydro-1H-purin-8-yl]thio}butanoate; ethyl 2-{[7-(3-bromobenzyl)-1-(3-hydroxypropyl)-3-methyl-2,6-dioxo-2,3,6,7-tetrahydro-1H-purin-8-yl]thio}butanoate; ethyl 2-{[7-(3-bromobenzyl)-3-methyl-2,6-dioxo-1-(2-propynyl)-2,3,6,7-tetrahydro-1H-purin-8-yl]thio}butanoate; ethyl 2-{[7-(3-methoxybenzyl)-3-methyl-2,6-dioxo-2,3,6,7-tetrahydro-1H-purin-8-yl]thio}butanoate; ethyl 2-{[3-methyl-7-(3-nitrobenzyl)-2,6-dioxo-2,3,6,7-tetrahydro-1H-purin-8-yl]thio}butanoate; ethyl 2-{[7-(3-aminobenzyl)-3-methyl-2,6-dioxo-2,3,6,7-tetrahydro-1H-purin-8-yl]thio}butanoate; ethyl 2-({7-[4-(aminosulfonyl)benzyl]-3-methyl-2,6-dioxo-2,3,6,7-tetrahydro-1H-purin-8-yl}thio)butanoate; ethyl 2-{[7-(4-bromobenzyl)-1,3-dimethyl-2,6-dioxo-2,3,6,7-tetrahydro-1H-purin-8-yl]thio}butanoate; ethyl 2-{[7-(cyclohexylmethyl)-1,3-dimethyl-2,6-dioxo-2,3,6,7-tetrahydro-1H-purin-8-yl]thio}butanoate; ethyl 2-{[1,3-dimethyl-2,6-dioxo-7-(1-phenylethyl)-2,3,6,7-tetrahydro-1H-purin-8-yl]thio}butanoate; ethyl 2-{[1,3-dimethyl-2,6-dioxo-7-(2-phenylethyl)-2,3,6,7-tetrahydro-1H-purin-8-yl]thio}butanoate; ethyl 2-({7-[(3,5-dimethylisoxazol-4-yl)methyl]-3-methyl-2,6-dioxo-2,3,6,7-tetrahydro-1H-purin-8-yl}thio)butanoate; ethyl 2-({3-methyl-7-[(5-nitro-2-furyl)methyl]-2,6-dioxo-2,3,6,7-tetrahydro-1H-purin-8-yl}thio)butanoate; ethyl 2-[(7-butyl-3-methyl-2,6-dioxo-2,3,6,7-tetrahydro-1H-purin-8-yl)thio]butanoate; ethyl 2-{[7-(3-bromobenzyl)-2,6-dioxo-2,3,6,7-tetrahydro-1H-purin-8-yl]thio}butanoate; ethyl 2-[(1,7-dihexyl-3-methyl-2,6-dioxo-2,3,6,7-tetrahydro-1H-purin-8-yl)thio]butanoate; ethyl 2-[(7-hexyl-3-methyl-2,6-dioxo-2,3,6,7-tetrahydro-1H-purin-8-yl)thio]butanoate; ethyl 2-[(3-methyl-2,6-dioxo-1,7-dipentyl-2,3,6,7-tetrahydro-1H-purin-8-yl)thio]butanoate; 2-{[7-(3-bromobenzyl)-3-methyl-2,6-dioxo-2,3,6,7-tetrahydro-1H-purin-8-yl]thio}butanamide; 2-[(7-butyl-3-methyl-2,6-dioxo-2,3,6,7-tetrahydro-1H-purin-8-yl)thio]butanamide; 7-(3-bromobenzyl)-8-[(1-ethylpropyl)thio]-3-methyl-3,7-dihydro-1H-purine-2,6-dione; ethyl 2-{8-[(3-bromobenzyl)thio]-1,3-dimethyl-2,6-dioxo-1,2,3,6-tetrahydro-7H-purin-7-yl}butanoate; and ethyl 2-[(7-isobutyl-3-methyl-2,6-dioxo-2,3,6,7-tetrahydro-1H-purin-8-yl)thio]butanoate.

More preferred compounds are: ethyl 2-[(7-benzyl-1,3-dimethyl-2,6-dioxo-2,3,6,7-tetrahydro-1H-purin-8-yl)thio]butanoate; ethyl 2-{[7-(3-bromobenzyl)-1-(2-methoxyethyl)-3-methyl-2,6-dioxo-2,3,6,7-tetrahydro-1H-purin-8-yl]thio}butanoate; ethyl 2-{[7-(3-bromobenzyl)-1,3-dimethyl-2,6-dioxo-2,3,6,7-tetrahydro-1H-purin-8-yl]thio}butanoate; ethyl 2-{[7-(3-bromobenzyl)-1-(cyanomethyl)-3-methyl-2,6-dioxo-2,3,6,7-tetrahydro-1H-purin-8-yl]thio}butanoate; ethyl 2-{[7-(3-bromobenzyl)-3-methyl-2,6-dioxo-1-propyl-2,3,6,7-tetrahydro-1H-purin-8-yl]thio}butanoate; ethyl 2-{[7-(3-bromobenzyl)-1-(3-hydroxypropyl)-3-methyl-2,6-dioxo-2,3,6,7-tetrahydro-1H-purin-8-yl]thio}butanoate; ethyl 2-{[7-(3-bromobenzyl)-3-methyl-2,6-dioxo-1-(2-propynyl)-2,3,6,7-tetrahydro-1H-purin-8-yl]thio}butanoate; ethyl 2-{[7-(3-methoxybenzyl)-3-methyl-2,6-dioxo-2,3,6,7-tetrahydro-1H-purin-8-yl]thio}butanoate; ethyl 2-{[3-methyl-7-(3-nitrobenzyl)-2,6-dioxo-2,3,6,7-tetrahydro-1H-purin-8-yl]thio}butanoate; ethyl 2-{[7-(3-aminobenzyl)-3-methyl-2,6-dioxo-2,3,6,7-tetrahydro-1H-purin-8-yl]thio}butanoate; ethyl 2-({7-[(3,5-dimethylisoxazol-4-yl)methyl]-3-methyl-2,6-dioxo-2,3,6,7-tetrahydro-1H-purin-8-yl}thio)butanoate; ethyl 2-({3-methyl-7-[(5-nitro-2-furyl)methyl]-2,6-dioxo-2,3,6,7-tetrahydro-1H-purin-8-yl}thio)butanoate; ethyl 2-[(7-butyl-3-methyl-2,6-dioxo-2,3,6,7-tetrahydro-1H-purin-8-yl)thio]butanoate; ethyl 2-[(7-hexyl-3-methyl-2,6-dioxo-2,3,6,7-tetrahydro-1H-purin-8-yl)thio]butanoate; 2-{[7-(3-bromobenzyl)-3-methyl-2,6-dioxo-2,3,6,7-tetrahydro-1H-purin-8-yl]thio}butanamide; 7-(3-bromobenzyl)-8-[(1-ethylpropyl)thio]-3-methyl-3,7-dihydro-1H-purine-2,6-dione; and ethyl 2-{8-[(3-bromobenzyl)thio]-1,3-dimethyl-2,6-dioxo-1,2,3,6-tetrahydro-7H-purin-7-yl}butanoate.

Most preferred compounds are: ethyl 2-{[7-(3-methoxybenzyl)-3-methyl-2,6-dioxo-2,3,6,7-tetrahydro-1H-purin-8-yl]thio}butanoate; ethyl 2-{[3-methyl-7-(3-nitrobenzyl)-2,6-dioxo-2,3,6,7-tetrahydro-1H-purin-8-yl]thio}butanoate; and ethyl 2-({3-methyl-7-[(5-nitro-2-furyl)methyl]-2,6-dioxo-2,3,6,7-tetrahydro-1H-purin-8-yl}thio)butanoate.

The “pharmaceutically acceptable salts” according to the invention include therapeutically active, non-toxic acid or base salt forms which the compounds of formula I are able to form.

The acid addition salt form of a compound of formula I that occurs in its free form as a base can be obtained by treating the free base with an appropriate acid such as an inorganic acid, for example, a hydrohalic such as hydrochloric or hydrobromic, sulfuric, nitric, phosphoric and the like; or an organic acid, such as, for example, acetic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, pamoic and the like.

The compounds of formula I containing acidic protons may be converted into their therapeutically active, non-toxic base addition salt forms, e.g. metal or amine salts, by treatment with appropriate organic and inorganic bases. Appropriate base salt forms include, for example, ammonium salts, alkali and earth alkaline metal salts, e.g. lithium, sodium, potassium, magnesium, calcium salts and the like, salts with organic bases, e.g. N-methyl-D-glucamine, hydrabamine salts, and salts with amino acids such as, for example, arginine, lysine and the like.

Conversely said salt forms can be converted into the free forms by treatment with an appropriate base or acid.

Compounds of the formula I and their salts can be in the form of a solvate, which is included within the scope of the present invention. Such solvates include for example hydrates, alcoholates and the like.

Many of the compounds of formula I and some of their intermediates have at least one stereogenic center in their structure. This stereogenic center may be present in a R or a S configuration, said R and S notation is used in correspondence with the rules described in Pure Appl. Chem., 45 (1976) 11-30.

The invention also relates to all stereoisomeric forms such as enantiomeric and diastereoisomeric forms of the compounds of formula I or mixtures thereof (including all possible mixtures of stereoisomers).

With respect to the present invention reference to a compound or compounds is intended to encompass that compound in each of its possible isomeric forms and mixtures thereof, unless the particular isomeric form is referred to specifically.

Compounds according to the present invention may exist in different polymorphic forms. Although not explicitly indicated in the above formula, such forms are intended to be included within the scope of the present invention.

The compounds of formula I according to the invention can be prepared analogously to conventional methods as understood by the person skilled in the art of synthetic organic chemistry.

According to one embodiment, some compounds having the general formula I may be prepared by alkylation of a compound of formula III with a compound of formula IV according to the equation

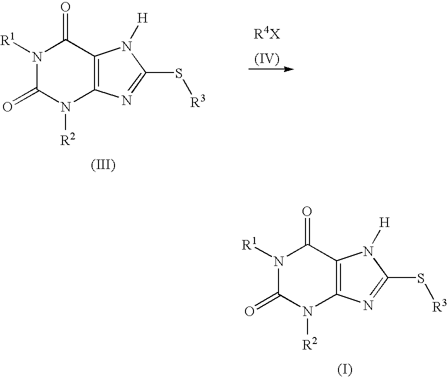

wherein X is an halogen atom, preferably bromo or chloro.

This reaction may be carried out according to any method known to the person skilled in the art.

Compounds of formula III may be prepared by reaction of a compound of formula V with one equivalent of an alkyl halide of formula VI according to the equation

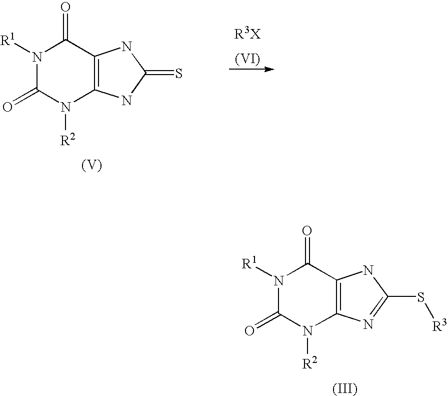

This reaction may be carried out in DMF (N,N dimethylformamide) at 25° C. in the presence of potassium carbonate providing selectively the corresponding S-alkylated product of formula III.

Compounds of formula V may be synthesized by a three-step procedure according to the equation

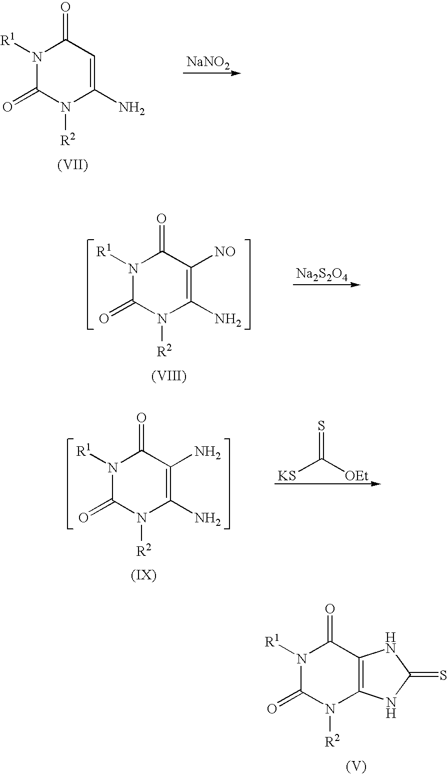

This three-step procedure consists in the nitrosation of a 6-aminouracil of formula (VII), followed by a sodium dithionite reduction of the nitroso function of intermediate (VIII), then by a ring closure of intermediate (IX) using potassium ethyl xanthate, as described by H. B. Cottam and al. in J. Med. Chem. (1996), 39, 2-9.

In another embodiment, some compounds having the general formula I wherein R¹ is different from hydrogen may be prepared by alkylation of the corresponding compound of formula I wherein R¹ is hydrogen according to the equation

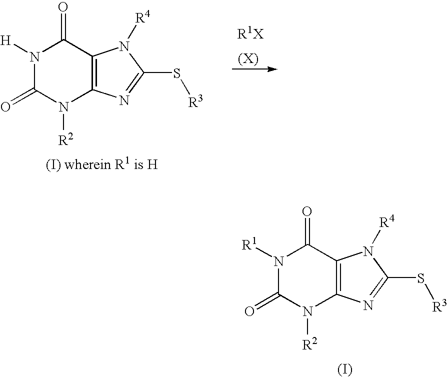

This reaction may be carried out according to any method known to the person skilled in the art.

In another embodiment, some compounds having the general formula I wherein R³ is —CHR⁵R⁶ and R⁶ is —CONH₂ may be prepared by ammonolysis, in methanol, of the corresponding ester of formula I wherein R⁶ is —COOR⁷, R⁷ being a C₁₋₄ alkyl.

In one embodiment, the present invention concerns also the synthesis of intermediate compounds of formula III

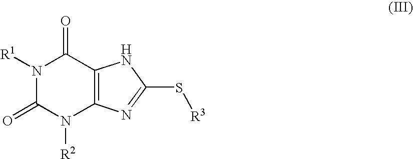

wherein

R¹ is hydrogen or C₁₋₆ alkyl;

R² is hydrogen or C₁₋₄ alkyl;

R³ is a group of formula —CHR⁵R⁶ or a benzyl group;

R⁵ is C₂₋₄ alkyl;

R⁶ is C₂₋₄ alkyl, amido or —COOR⁷;

R⁷ is C₁₋₄ alkyl.

In a preferred embodiment, the present invention concerns also the synthesis intermediates of formula III selected from the group of ethyl 2-[(1,3-dimethyl-2,6-dioxo-2,3,6,7-tetrahydro-1H-purin-8-yl)thio]butanoate; ethyl 2-[(2,6-dioxo-2,3,6,7-tetrahydro-1H-purin-8-yl)thio]butanoate; ethyl 2-[(3-methyl-2,6-dioxo-2,3,6,7-tetrahydro-1H-purin-8-yl)thio]butanoate; 8-[(1-ethylpropyl)thio]-3-methyl-3,7-dihydro-1H-purine-2,6-dione; and 8-[(3-bromobenzyl)thio]-3-methyl-3,7-dihydro-1H-purine-2,6-dione.

It has now been found that compounds of formula II, their enantiomers, diastereoisomers and mixtures thereof (including all possible mixtures of stereoisomers), or pharmaceutically acceptable salts

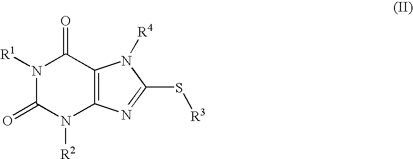

wherein

R¹ is hydrogen or C₁₋₆ alkyl;

R² is hydrogen or C₁₋₄ alkyl;

R³ is a group of formula —CHR⁵R⁶ or a benzyl group;

R⁴ is C₁₋₈ alkyl optionally substituted by alkoxycarbonyl, C₃₋₆ cycloalkyl, aryl or heterocycle;

R⁵ is hydrogen or C₁₋₄ alkyl;

R⁶ is C₁₋₄ alkyl, amido or —COOR⁷;

R⁷ is C₁₋₄ alkyl;

are useful in a variety of therapeutic disorders.

In the above embodiment, preferably, when R³ is a benzyl group, then R⁴ is C₁₋₈ alkyl optionally substituted by alkoxycarbonyl.

In the above embodiment, preferably, when R³ is a group of formula —CHR⁵R⁶, then R⁴ is C₁₋₈ alkyl optionally substituted by C₃₋₆ cycloalkyl, aryl or heterocycle.

For example, the compounds according to the invention are useful for the treatment of epilepsy, epileptogenesis, seizure disorders, incontinence and convulsions.

These compounds may also be used for the treatment of Parkinson's disease.

These compounds may also be used for the treatment of dyskinesia induced by dopamine replacement therapy, tardive dyskinesia induced by administration of neuroleptic drugs or Huntington Chorea.

In another aspect the invention therefore provides the therapeutical use of compounds of formula II, their enantiomers, diastereoisomers and mixtures thereof (including all possible mixtures of stereoisomers), or pharmaceutically acceptable salts

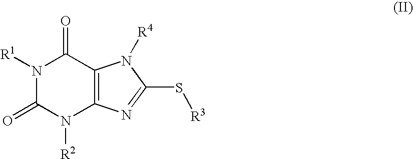

wherein

R¹ is hydrogen or C₁₋₆ alkyl;

R² is hydrogen or C₁₋₄ alkyl;

R³ is a group of formula —CHR⁵R⁶ or a benzyl group;

R⁴ is C₁₋₈ alkyl optionally substituted by alkoxycarbonyl, C₃₋₆ cycloalkyl, aryl or heterocycle;

R⁵ is hydrogen or C₁₋₄ alkyl;

R⁶ is C₁₋₄ alkyl, amido or —COOR⁷;

R⁷ is C₁₋₄ alkyl.

In a particular embodiment, the invention provides the therapeutical use of compounds of formula II selected from ethyl 2-[(7-heptyl-3-methyl-2,6-dioxo-2,3,6,7-tetrahydro-1H-purin-8-yl)thio]butanoate; 7-(3-bromobenzyl)-3-methyl-8-(propylthio)-3,7-dihydro-1H-purine-2,6-dione; ethyl 2-[(3-methyl-2,6-dioxo-7-pentyl-2,3,6,7-tetrahydro-1H-purin-8-yl)thio]butanoate; ethyl 2-{[7-(3-bromobenzyl)-3-methyl-2,6-dioxo-2,3,6,7-tetrahydro-1H-purin-8-yl]thio}butanoate; ethyl 2-[(3-methyl-2,6-dioxo-7-propyl-2,3,6,7-tetrahydro-1H-purin-8-yl)thio]butanoate; 7-(3-bromobenzyl)-8-[(3-chloro-2-hydroxypropyl)thio]-3-methyl-3,7-dihydro-1H-purine-2,6-dione; and ethyl 2-{[7-(3-bromobenzyl)-3-methyl-2,6-dioxo-2,3,6,7-tetrahydro-1H-purin-8-yl]thio}propanoate.

In another particular embodiment, the invention provides the therapeutical use of compounds of formula I, their enantiomers, diastereoisomers and mixtures thereof (including all possible mixtures of stereoisomers), or pharmaceutically acceptable salts

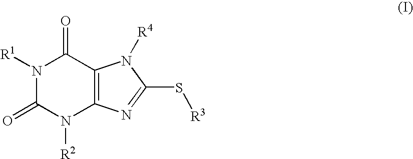

wherein

R¹ is hydrogen or C₁₋₆ alkyl;

R² is hydrogen or C₁₋₄ alkyl;

R³ is a group of formula —CHR⁵R⁶ or a benzyl group;

R⁴ is C₁₋₈ alkyl optionally substituted by alkoxycarbonyl, C₃₋₆ cycloalkyl, aryl or heterocycle;

R⁵ is C₂₋₄ alkyl;

R⁶ is C₂₋₄ alkyl, amido or —COOR⁷;

R⁷ is C₁₋₄ alkyl;

with the proviso that when R¹ is hydrogen, R² is methyl, R³ is —CHR⁵R⁶, R⁶ is ethoxycarbonyl and R⁵ is ethyl, then R⁴ is different from n-propyl, i-propyl, n-pentyl, n-heptyl, 3-bromobenzyl, 4-chlorobenzyl, 4-methylbenzyl or 2-phenylethyl.

In another embodiment, the invention concerns a pharmaceutical composition of compounds having formula II, their enantiomers, diastereoisomers and mixtures thereof (including all possible mixtures of stereoisomers), or pharmaceutically acceptable salts thereof,

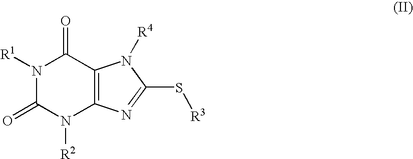

wherein

R¹ is hydrogen or C₁₋₆ alkyl;

R² is hydrogen or C₁₋₄ alkyl;

R³ is a group of formula —CHR⁵R⁶ or a benzyl group;

R⁴ is C₁₋₈ alkyl optionally substituted by alkoxycarbonyl, C₃₋₆ cycloalkyl, aryl or heterocycle;

R⁵ is hydrogen or C₁₋₄ alkyl;

R⁶ is C₁₋₄ alkyl, amido or —COOR⁷;

R⁷ is C₁₋₄ alkyl;

and a pharmaceutical carrier.

The present invention also concerns use of a compound having formula I or formula II for the manufacture of a medicament for the treatment and prevention of epilepsy, epileptogenesis, seizure disorders, convulsions, Parkinson's disease, dyskinesia induced by dopamine replacement therapy, tardive dyskinesia induced by administration of neuroleptic drugs, Huntington Chorea, and other neurological disorders including bipolar disorders, mania, depression, anxiety, attention deficit hyperactivity disorder (ADHD), migraine, trigeminal and other neuralgia, chronic pain, neuropathic pain, cerebral ischemia, cardiac arrhythmia, myotonia, cocaine abuse, stroke, myoclonus, tremor, essential tremor, simple or complex tics, Tourette syndrome, restless leg syndrome and other movement disorders, neonatal cerebral haemorrhage, amyotrophic lateral sclerosis, spasticity and degenerative diseases, bronchial asthma, asthmatic status and allergic bronchitis, asthmatic syndrome, bronchial hyperreactivity and bronchospastic syndromes as well as allergic and vasomotor rhinitis and rhinoconjunctivitis.

In addition, the compounds according to formulae I and II may also be used for treating other neurological disorders including bipolar disorders, mania, depression, anxiety, attention deficit hyperactivity disorder (ADHD), migraine, trigeminal and other neuralgia, chronic pain, neuropathic pain, cerebral ischemia, cardiac arrhythmia, myotonia, cocaine abuse, stroke, myoclonus, tremor, essential tremor, simple or complex tics, Tourette syndrome, restless leg syndrome and other movement disorders, neonatal cerebral haemorrhage, amyotrophic lateral sclerosis, spasticity and degenerative diseases, bronchial asthma, asthmatic status and allergic bronchitis, asthmatic syndrome, bronchial hyperreactivity and bronchospastic syndromes as well as allergic and vasomotor rhinitis and rhinoconjunctivitis.

Thus, the present invention also concerns a compound having formulae I or II or a pharmaceutically acceptable salt thereof as defined above for use as a medicament.

In a further aspect, the present invention concerns also the use of a compound of formulae I or II or a pharmaceutically acceptable salt thereof for the manufacture of a medicament for the treatment of neurological and other disorders such as mentioned above.

In particular, the present invention concerns the use of a compound of formulae I or II or a pharmaceutically acceptable salt thereof for the manufacture of a medicament for the treatment of epilepsy, Parkinson's disease, dyskinesia, migraine, tremor, essential tremor, bipolar disorders, chronic pain, neuropathic pain, or bronchial, asthmatic or allergic conditions.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 3 to 3000 mg, preferably 25 to 500 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The term “epilepsy” as used herein refers to a chronic neurologic condition characterised by unprovoked, recurrent epileptic seizures. An epileptic seizure is the manifestation of an abnormal and excessive synchronised discharge of a set of cerebral neurons; its clinical manifestations are sudden and transient. The term “epilepsy” as used herein can also refer to a disorder of brain function characterised by the periodic occurrence of seizures. Seizures can be “non-epileptic” when evoked in a normal brain by conditions such as high fever or exposure to toxins or “epileptic” when evoked without evident provocation.

The term “seizure” as used herein refers to a transient alteration of behaviour due to the disordered, synchronous, and rhythmic firing of populations of brain neurones.

The term “Parkinsonian symptoms” relates to a syndrome characterised by slowness of movement (bradykinesia), rigidity and/or tremor. Parkinsonian symptoms are seen in a variety of conditions, most commonly in idiopathic parkinsonism (i.e. Parkinson's Disease) but also following treatment of schizophrenia, exposure to toxins/drugs and head injury. It is widely appreciated that the primary pathology underlying Parkinson's disease is degeneration, in the brain, of the dopaminergic projection from the substantia nigra to the striatum. This has led to the widespread use of dopamine-replacing agents (e.g. L-3,4-dihydroxyphenylalanine (L-DOPA) and dopamine agonists) as symptomatic treatments for Parkinson's disease and such treatments have been successful in increasing the quality of life of patients suffering from Parkinson's disease. However, dopamine-replacement treatments do have limitations, especially following long-term treatment. Problems can include a wearing-off of the anti-parkinsonian efficacy of the treatment and the appearance of a range of side-effects which manifest as abnormal involuntary movements, such as dyskinesias.

The term “dyskinesia” is defined as the development in a subject of abnormal involuntary movements. This appears in patients with Huntington's disease, in Parkinson's disease patients exposed to chronic dopamine replacement therapy, and in Schizophrenia patients exposed to chronic treatment with neuroleptics. Dyskinesias, as a whole, are characterised by the development in a subject of abnormal involuntary movements. One way in which dyskinesias may arise is as a side effect of dopamine replacement therapy for parkinsonism or other basal ganglia-related movement disorders.

The term “migraine” as used herein means a disorder characterised by recurrent attacks of headache that vary widely in intensity, frequency, and duration. The attacks are commonly unilateral and are usually associated with anorexia, nausea, vomiting, phonophobia, and/or photophobia. In some cases they are preceded by, or associated with, neurological and mood disturbances. Migraine headache may last from 4 hours to about 72 hours. The International Headache Society (IHS, 1988) classifies migraine with aura (classical migraine) and migraine without aura (common migraine) as the major types of migraine. Migraine with aura consists of a headache phase preceded by characteristic visual, sensory, speech, or motor symptoms. In the absence of such symptoms, the headache is called migraine without aura.

The term “bipolar disorders” as used herein refers to those disorders classified as Mood Disorders according to the Diagnostic and Statistical Manual of Mental Disorders, 4th edition (Diagnostic and Statistical Manual of Mental Disorders (DSM-IV™), American Psychiatry Association, Washington, D.C., 1994). Bipolar disorders are generally characterised by spontaneously triggered repeated (i.e. at least two) episodes in which the patient's hyperexcitability, activity and mood are significantly disturbed, this disturbance consisting on some occasions of an elevation of mood and increased energy and activity (mania or hypomania), and in other occasions a lowering of mood and decreased energy and activity (depression). Bipolar disorders are separated into four main categories in the DSM-IV (bipolar I disorder, bipolar II disorder, cyclothymia, and bipolar disorders not otherwise specified).

The term “manic episode”, as used herein refers to a distinct period during which there is an abnormally and persistently elevated, expansive, or irritable mood with signs of pressured speech and psychomotor agitation.

The term “hypomania”, as used herein refers to a less extreme manic episode, with lower grade of severity.

The term “major depressive episode”, as used herein refers to a period of at least 2 weeks during which there is either depressed mood or the loss of interest or pleasure in nearly all activities with signs of impaired concentration and psychomotor retardation.

The term “mixed episode”, as used herein refers to a period of time (lasting at least 1 week) in which the criteria are met both for a manic episode and for a major depressive episode nearly every day.

The term “chronic pain” as used herein refers to the condition gradually being recognised as a disease process distinct from acute pain. Conventionally defined as pain that persists beyond the normal time of healing, pain can also be considered chronic at the point when the individual realises that the pain is going to be a persistent part of their lives for the foreseeable future. It is likely that a majority of chronic pain syndromes involves a neuropathic component, which is usually harder to treat than acute somatic pain.

The term “neuropathic pain” as used herein refers to pain initiated by a pathological change in a nerve which signals the presence of a noxious stimulus when no such recognisable stimulus exists, giving rise to a false sensation of pain. In other words, it appears that the pain system has been turned on and cannot turn itself off.

The term “tics” refers to common and often disabling neurological disorders. They are frequently associated with behaviour difficulties, including obsessive-compulsive disorder, attention deficit hyperactivity disorder and impulse control. Tics are involuntary, sudden, rapid, repetitive, nonrhythmic stereotype movements or vocalizations. Tics are manifested in a variety of forms, with different durations and degrees of complexity. Simple motor tics are brief rapid movements that often involve only one muscle group. Complex motor tics are abrupt movements that involve either a cluster of simple movements or a more coordinated sequence of movements. Simple vocal tics include sounds such as grunting, barking, yelping, and that clearing. Complex vocal tics include syllables, phrases, repeating other people's words and repeating one's own words.

An assay indicative of potential anticonvulsant activity is binding to levetiracetam binding site (LBS) as hereinafter described. As set forth in U.S. patent application Ser. Nos. 10/308,163 and 60/430,372 LBS has been identified as belonging to the family of SV2 proteins. As used herein reference to “LBS” is to be understood as including reference to SV2.

Activity in any of the above-mentioned indications can of course be determined by carrying out suitable clinical trials in a manner known to a person skilled in the relevant art for the particular indication and/or in the design of clinical trials in general.

For treating diseases, compounds of formula I or their pharmaceutically acceptable salts may be employed at an effective daily dosage and administered in the form of a pharmaceutical composition.

Therefore, another embodiment of the present invention concerns a pharmaceutical composition comprising an effective amount of a compound of formulae I or II or a pharmaceutically acceptable salt thereof in combination with a pharmaceutically acceptable diluent or carrier.

To prepare a pharmaceutical composition according to the invention, one or more of the compounds of formulae I or II or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral or intranasal.

Pharmaceutical compositions comprising compounds according to the invention can, for example, be administered orally, parenterally, i.e., intravenously, intramuscularly or subcutaneously, intrathecally, by inhalation or intranasally.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula I in compositions for oral administration is at least 0.5% by weight and can be up to 80% by weight with respect to the total weight of the composition.

In accordance with the invention it has also been found that the compounds of formulae I or II or the pharmaceutically acceptable salts thereof can be administered alone or in combination with other pharmaceutically active ingredients. Non-limiting examples of such additional compounds which can be cited for use in combination with the compounds according to the invention are antivirals, antispastics (e.g. baclofen), antiemetics, antimanic mood stabilizing agents, analgesics (e.g. aspirin, ibuprofen, paracetamol), narcotic analgesics, topical anesthetics, opioid analgesics, lithium salts, antidepressants (e.g. mianserin, fluoxetine, trazodone), tricyclic antidepressants (e.g. imipramine, desipramine), anticonvulsants (e.g. valproic acid, carbamazepine, phenyloin), antipsychotics (e.g. risperidone, haloperidol), neuroleptics, benzodiazepines (e.g. diazepam, clonazepam), phenothiazines (e.g. chlorpromazine), calcium channel blockers, amphetamine, clonidine, lidocaine, mexiletine, capsaicin, caffeine, quetiapine, serotonin antagonists, β-blockers, antiarrhythmics, triptans, ergot derivatives and amantadine.

Of particular interest in accordance with the present invention are combinations of at least one compound of formulae I or II or a pharmaceutically acceptable salt thereof and at least one compound inducing neural inhibition mediated by GABA_{A} receptors. The compounds of formulae I or II exhibit a potentiating effect on the compounds inducing neural inhibition mediated by GABA_{A} receptors enabling, in many cases, effective treatment of conditions and disorders under reduced risk of adverse effects.

Examples of compounds inducing neural inhibition mediated by GABA_{A} receptors include the following: benzodiazepines, barbiturates, steroids, and anticonvulsants such as valproate, viagabatrine, tiagabine or pharmaceutical acceptable salts thereof.

Benzodiazepines include the 1,4-benzodiazepines, such as diazepam and clonazepam, and the 1,5-benzodiazepines, such as clobazam. Preferred compound is clonazepam.

Barbiturates include phenobarbital and pentobarbital. Preferred compound is phenobarbital.

Steroids include adrenocorticotropic hormones such as tetracosactide acetate, etc.

Anticonvulsants include hydantoins (phenyloin, ethotoin, etc), oxazoiidines (trimethadione, etc.), succinimides (ethosuximide, etc.), phenacemides (phenacemide, acetylpheneturide, etc.), sulfonamides (sulthiame, acetoazolamide, etc.), aminobutyric acids (e.g. gamma-amino-beta-hydroxybutyric acid, etc.), sodium valproate and derivatives, carbamazepine and so on.

Preferred compounds include valproic acid, valpromide, valproate pivoxil, sodium valproate, semi-sodium valproate, divalproex, clonazepam, phenobarbital, vigabatrine, tiagabine, amantadine.

For the preferred oral compositions, the daily dosage is in the range 3 to 3000 milligrams (mg) of compounds of formulae I or II.

In compositions for parenteral administration, the quantity of compound of formula I present is at least 0.5% by weight and can be up to 33% by weight with respect to the total weight of the composition. For the preferred parenteral compositions, the dosage unit is in the range 3 mg to 3000 mg of compounds of formula I or II.

The daily dose can fall within a wide range of dosage units of compound of formula I and is generally in the range 3 to 3000 mg. However, it should be understood that the specific doses can be adapted to particular cases depending on the individual requirements, at the physician's discretion.

The LBS binding compounds provided by this invention and labelled derivatives thereof may be useful as standards and reagents in determining the ability of tested compounds (e.g., a potential pharmaceutical) to bind to the LBS receptor.

Labelled derivatives of LBS ligands provided by this invention may also be useful as radiotracers for positron emission tomography (PET) imaging or for single photon emission computerized tomography (SPECT).

The present invention therefore further provides labelled ligands as tools to screen chemical libraries for the discovery of potential pharmaceutical agents, in particular for treatment and prevention of the conditions set forth herein, on the basis of more potent binding to LBS/SV2 proteins, for localizing SV2 proteins in tissues, and for characterizing purified SV2 proteins. SV2 proteins include SV2A, SV2B, and SV2C whereby SV2A is the binding site for the anti-seizure drug levetiracetam and its analogs. The SV2 isoforms SV2A, SV2B, or SV2C can be derived from tissues, especially brain, from any mammal species, including human, rat or mice. Alternately the isoforms may be cloned versions of any mammalian species, including human, rat, and mice, heterologously expressed and used for assays. The screening method comprises exposing brain membranes, such as mammalian or human brain membranes, or cell lines expressing SV2 proteins or fragments thereof, especially SV2A, but including SV2B and SV2C, to a putative agent and incubating the membranes or proteins or fragments and the agent with labelled compound of formulae I or II. The method further comprises determining if the binding of the compound of formulae I or II to the protein is inhibited by the putative agent, thereby identifying binding partners for the protein. Thus, the screening assays enable the identification of new drugs or compounds that interact with LBS/SV2. The present invention also provides photoactivable ligands of SV2/LBS.

The labelled-ligands can also be used as tools to assess the conformation state of SV2 proteins after solubilization, purification and chromatography. The labelled-ligands may be directly or indirectly labeled. Examples of suitable labels include a radiolabel, such as ³H, a fluorescent label, an enzyme, europium, biotin and other conventional labels for assays of this type.

Screening assays of the present invention include methods of identifying agents or compounds that compete for binding to the LBS (especially SV2A). Labelled compounds of formulae I or II are useful in the methods of the invention as probes in assays to screen for new compounds or agents that bind to the LBS (especially SV2A). In such assay embodiments, ligands can be used without modification or can be modified in a variety of ways; for example, by labelling, such as covalently or non-covalently joining a moiety which directly or indirectly provides a detectable signal. In any of these assays, the materials can be labelled either directly or indirectly. Possibilities for direct labelling include label groups such as: radiolabels including, but not limited to, [³H], [¹⁴C], [³²P], [³⁵S] or [¹²⁵I], enzymes such as peroxidase and alkaline phosphatase, and fluorescent labels capable of monitoring the change in fluorescence intensity, wavelength shift, or fluorescence polarization, including, but not limited to, fluorescein or rhodamine. Possibilities for indirect labelling include biotinylation of one constituent followed by binding to avidin coupled to one of the above label groups or the use of anti-ligand antibodies. The compounds may also include spacers or linkers in cases where the compounds are to be attached to a solid support. To identify agents or compounds which compete or interact with labelled ligands according to the invention for binding to the LBS (especially SV2A), intact cells, cellular or membrane fragments containing SV2A or the entire SV2 protein or a fragment comprising the LBS of the SV2 protein can be used. The agent or compound may be incubated with the cells, membranes, SV2 protein or fragment prior to, at the same time as, or after incubation with levetiracetam or an analog or derivative thereof. Assays of the invention may be modified or prepared in any available format, including high-throughput screening (HTS) assays that monitor the binding of levetiracetam or the binding of derivatives or analogs thereof to SV2 or to the LBS of the SV2 protein. in many drug screening programs which test libraries of compounds, high throughput assays are desirable in order to maximize the number of compounds surveyed in a given period of time. Such screening assays may use intact cells, cellular or membrane fragments containing SV2 as well as cell-free or membrane-free systems, such as may be derived with purified or semi-purified proteins. The advantage of the assay with membrane fragment containing SV2 or purified SV2 proteins and peptides is that the effects of cellular toxicity and/or bioavailability of the test compound can be generally ignored, the assay instead being focused primarily on the effect of the drug on the molecular target as may be manifest in an inhibition of, for instance, binding between two molecules. The assay can be formulated to detect the ability of a test agent or compound to inhibit binding of labelled ligand according to the invention to SV2 or a fragment of SV2 comprising the LBS or of levetiracetam, or derivatives or analogs thereof, to SV2 or a fragment of SV2 comprising the LBS. The inhibition of complex formation may be detected by a variety of techniques such as filtration assays, Flashplates (Perkin Elmer, scintillation proximity assays (SPA, Amersham Biosciences). For high-throughput screenings (HTS), scintillation proximity assay is a powerful method which uses microspheres coated with biological membranes and requires no separation or washing steps.

Labelled ligands are also useful for assessing the conformational state of SV2 after solubilization, purification, and chromatography. Moreover, the present invention provides photoactivable versions of the ligands for labelling and detection in biological samples. The photoactivable ligands may also be used to localize and purify SV2 from tissues, isolated cells, subcellular fractions and membranes. The photoactivable could also be used for SV2 cross-linking and identification of binding domains of LBS ligands.

The following examples are provided for illustrative purposes.

Unless specified otherwise in the examples, characterization of the compounds is performed according to the following methods:

¹H and ¹³C NMR spectra are recorded on an Advance 300 Brucker spectrometer (at 300.13 and 75.47 MHz respectively) with Me₄Si as an internal standard or on a BRUKER AC 250 Fourier Transform NMR Spectrometer fitted with an Aspect 3000 computer and a 5 mm ¹H/¹³C dual probehead or BRUKER DRX 400 FT NMR fitted with a SG Indigo² computer and a 5 mm inverse geometry ¹H/¹³C/¹⁵N triple probehead. The compound is studied in d₆-DMSO (or CDCl₃) solution at a probe temperature of 313 K or 300 K and at a concentration of 20 mg/ml. The instrument is locked on the deuterium signal of d₆-DMSO (or CDCl₃). Chemical shifts are given in ppm downfield from TMS taken as internal standard.

HPLC analyses are performed using one of the following systems:
an Agilent 1100 series HPLC system mounted with an INERTSIL ODS 3 C18, DP 5 μm, 250×4.6 mm column. The gradient ran from 100% solvent A (acetonitrile, water, H₃PO₄ (5/95/0.001, v/v/v)) to 100% solvent B (acetonitrile, water, H₃PO₄ (95/5/0.001, v/v/v)) in 6 min with a hold at 100% B of 4 min. The flow rate is set at 2.5 ml/min. The chromatography is carried out at 35° C.
a HP 1090 series HPLC system mounted with a HPLC Waters Symetry C18, 250×4.6 mm column. The gradient ran from 100% solvent A (MeOH, water, H₃PO₄ (15/85/0.001M, v/v/M)) to 100% solvent B (MeOH, water, H₃PO₄ (85/15/0.001 M, v/v/M)) in 10 min with a hold at 100% B of 10 min. The flow rate is set at 1 ml/min. The chromatography is carried out at 40° C.

Mass Spectrometric Measurements in LC/MS Mode are Performed as Follows:

HPLC Conditions

Analyses are performed using a WATERS Alliance HPLC system mounted with an INERTSIL ODS 3, DP 5 μm, 250×4.6 mm column.

The gradient ran from 100% solvent A (acetonitrile, water, TFA (trifluoroacetic acid) (10/90/0.1, v/v/v)) to 100% solvent B (acetonitrile, water, TFA (90/10/0.1, v/v/v)) in 7 min with a hold at 100% B of 4 min. The flow rate is set at 2.5 ml/min and a split of 1/25 is used just before API source.

MS Conditions

Samples are dissolved in acetonitrile/water, 70/30, v/v at the concentration of about 250 μgr/ml. API spectra (+ or −) are performed using a FINNIGAN (San Jose, Calif., USA) LCQ ion trap mass spectrometer. APCI source operated at 450° C. and the capillary heater at 160° C. ESI source operated at 3.5 kV and the capillary heater at 210° C.

Electron spray ionization mass spectra are obtained using a Micromass Quattro II mass spectrometer with capillary and cone voltages of 3.5 kV and 30 V respectively and source temperature of 60° C.

Melting points are determined in open glass capillaries using a Mettler FP1 apparatus or a Büchi 535 or 545 Tottoli-type fusionometre, and are not corrected, or by the onset temperature on a Perkin Elmer DSC 7.

Column chromatography is performed on silica gel 60 (70-230 mesh, Merck). Preparative chromatographic separations are performed on silicagel 60 Merck, particle size 15-40 μm, reference 1.15111.9025, using Novasep axial compression columns (80 mm i.d.); flow rates between 70 and 150 ml/min. Amount of silicagel and solvent mixtures as described in individual procedures.

Preparative Chiral Chromatographic separations are performed on a DAICEL Chiralpak AD 20 μm, 100*500 mm column using an in-house build instrument with various mixtures of lower alcohols and C5 to C8 linear, branched or cyclic alkanes at ±350 ml/min. Solvent mixtures as described in individual procedures.

The following examples illustrate how the compounds covered by formula (I) can be synthesized.

### EXAMPLE 1

### Synthesis of ethyl 2-[(7-benzyl-1,3-dimethyl-2,6-dioxo-2,3,6,7-tetrahydro-1H-purin-8-yl)thio]butanoate 1

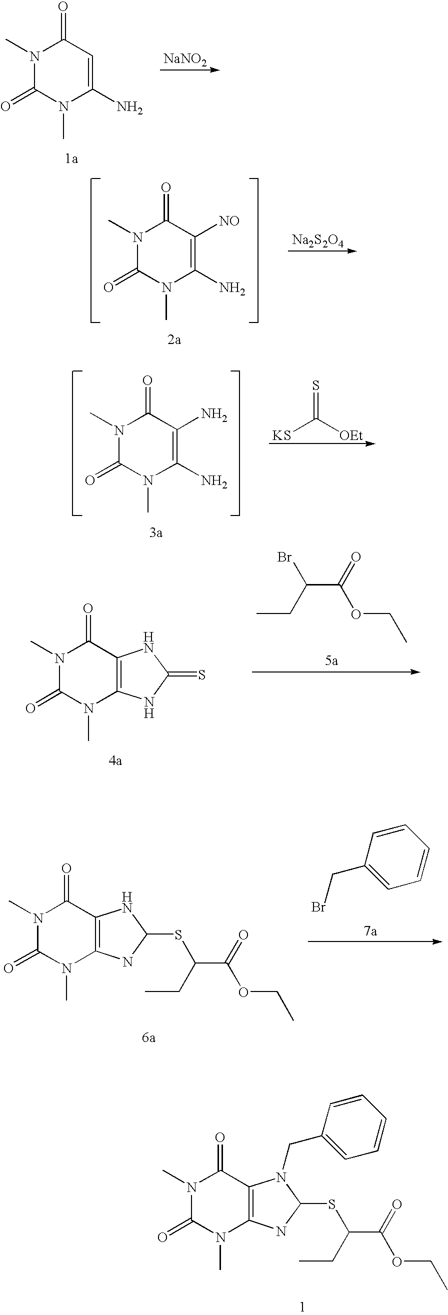

### 1.1 Synthesis of 1,3-dimethyl-8-thioxo-3,7,8,9-tetrahydro-1H-purine-2,6-dione 4a

Nitrosation: an aqueous solution of sodium nitrite (238 mmol in 100 ml) is added dropwise (30 min.) to a suspension of 6-amino-1,3-dimethylpyrimidine-2,4(1H,3H)-dione 1a (178 mmol) in 350 ml of 1N HCl. The suspension goes from off-white to purple almost immediately. Stirring is continued for 2 hours and the pH is adjusted to 7 by addition of concentrated ammonia (20 ml). The solid is then filtered, washed twice with water (50 ml) and used without drying in the next step.

Reduction: the wet 6-amino-1,3-dimethyl-5-nitrosopyrimidine-2,4(1H,3H)-dione 2a is suspended in 500 ml of water and heated to 85° C. Sodium dithionite (532 mmol) is added with stirring in portions over 40 min. The suspension changes from purple to green. The mixture is stirred at 85° C. for an additional 15 min., cooled to 0° C. and stirred 30 min. The precipitate is filtered, washed with cold water (4×30 ml), ethanol (2×30 ml) and diethylether (2×50 ml), and used without drying in the next step.

Ring Closure: a suspension of the wet 5,6-diamino-1,3-dimethylpyrimidine-2,4(1H,3H)-dione 3a and potassium ethyl xanthate (355 mmol) in DMF (750 ml) is heated at 100° C. for 2 hours. After cooling at room temperature, the precipitate is filtered and washed with diethylether (4×40 ml). The solid is dissolved in water (1 l) at 75° C. and the pH is adjusted to 4-5 by addition of glacial acetic acid (20 ml). A white precipitate appears, which is filtered at 40° C., washed with water (2×30 ml), ethanol (2×30 ml) and dried 15 hours under vacuum at room temperature to afford 1,3-dimethyl-8-thioxo-3,7,8,9-tetrahydro-1H-purine-2,6-dione 4a.

Yield: 32%.

Mp: >300° C.

MS (ES⁺): 213 (MH⁺).

¹H NMR (d₆-DMSO): 3.16 (s, 3H, NCH₃), 3.35 (s, 3H, NCH₃), 12.94 (m, 2H, NH).

The following compounds may be synthesized according to the same method:

| | | |
|---|---|---|
| 4b | 3-methyl-8-thioxo-3,7,8,9- | MS (ES⁺): 199 (MH⁺). |
| | tetrahydro-1H-purine-2,6-dione | ¹H NMR (d₆-DMSO): 3.30 (s, 3H, NCH₃), 11.20 |
| | | (s (broad), 1H, N¹H), 12.93 (m, 2H, NH). |
| 4c | 8-thioxo-3,7,8,9-tetrahydro-1H- | ¹³C NMR (d₆-DMSO): 103.6 (C⁵), 139.1 (C⁴), |
| | purine-2,6-dione | 150.1 (C²), 152.4 (C⁶), 163.7 (C⁸). |

### 1.2 Synthesis of ethyl 2-[(1,3-dimethyl-2,6-dioxo-2,3,6,7-tetrahydro-1H-purin-8-yl)thio]butanoate 6a

A suspension of 1,3-dimethyl-8-thioxo-3,7,8,9-tetrahydro-1H-purine-2,6-dione 4a (20.2 mmol), potassium carbonate (20.2 mmol) and ethyl 2-bromobutanoate 5a (20.2 mmol) in DMF (40 ml) is stirred at room temperature for 4 hours. The product is precipitated by addition of water (160 ml) and the pH is adjusted to 5-6 by addition of glacial acetic acid (4 ml). The mixture is stirred at 0° C. for 1 hour, filtered and washed with water (2×10 ml) and diethylether (3×10 ml). The solid is then suspended in diethylether (8 ml for 1 g), stirred for 1 hour at room temperature, filtered and washed with diethylether to afford ethyl 2-[(1,3-dimethyl-2,6-dioxo-2,3,6,7-tetrahydro-1H-purin-8-yl)thio]butanoate 6a.

Yield: 85%.

mp: 175° C.

MS (ES⁺): 327 (MH⁺).

¹H NMR (d₆-DMSO): 0.99 (t, J=7.3 Hz, 3H, SCHCH₂CH₃), 1.16 (t, J=7.4 Hz, 3H, OCH₂CH₃), 1.90 (m, 2H, SCHCH₂CH₃), 3.22 (s, 3H, NCH₃), 3.40 (s, 3H, NCH₃), 4.13 (q, J=7.4 Hz, 2H, OCH₂CH₃), 4.32 (t, J=6.9 Hz, 1H, SCHCH₂CH₃), 13.7 (s, 1H, NH).

The following compounds may be synthesized according to the same method:

| | | |
|---|---|---|
| 6b | ethyl 2-[(2,6-dioxo-2,3,6,7-tetrahydro-1H- | MS (ES⁺): 299 (MH⁺). |
| | purin-8-yl)thio]butanoate | ¹H NMR (d₆-DMSO): 0.93 (t, J = 7.3 Hz, 3H, |
| | | SCHCH₂CH₃), 1.17 (t, J = 7.1 Hz, 3H, |
| | | OCH₂CH₃), 1.83 (m, 2H, SCHCH₂CH₃), |
| | | 4.09 (q, J = 7.1 Hz, 2H, OCH₂CH₃), 4.23 (t, |
| | | J = 7.1 Hz, 1H, SCHCH₂CH₃), 9.74 and |
| | | 10.70 (m, 3H, NH). |
| 6c | ethyl 2-[(3-methyl-2,6-dioxo-2,3,6,7- | MS (ES⁺): 313 (MH⁺). |
| | tetrahydro-1H-purin-8-yl)thio]butanoate | ¹H NMR (d₆-DMSO): 0.98 (t, J = 7.4 Hz, 3H, |
| | | CHCH₂CH₃), 1.15 (t, J = 7.1 Hz, 3H, |
| | | OCH₂CH₃), 1.90 (m, 2H, CHCH₂CH₃), 3.33 |
| | | (s, 3H, NCH₃), 4.12 (q, J = 7.1 Hz, 2H, |
| | | OCH₂CH₃), 4.29 (t, J = 6.9 Hz, 1H, SCH), |
| | | 11.04 (s (broad), 1H, N¹H), 13.67 (s |
| | | (broad), 1H, N⁷H). |
| 6d | 8-[(1-ethylpropyl)thio]-3-methyl-3,7- | MS (ES⁺): 269 (MH⁺). |
| | dihydro-1H-purine-2,6-dione | ¹H NMR (d₆-DMSO): 0.96 (t, J = 7.2 Hz, 6H, |
| | | 2xCH₂CH₃), 1.66 (m, 4H, 2xCH₂CH₃), 3.34 |
| | | (s, 3H, N³CH₃), 3.61 (m, 1H, SCH), 11.03 |
| | | (s (broad), 1H, N¹H), 13.49 (s (broad), 1H, |
| | | N⁷H) |
| 6e | 8-[(3-bromobenzyl)thio]-3-methyl-3,7- | ¹H NMR (d₆-DMSO): 3.21 (s, 3H, NCH₃), |
| | dihydro-1H-purine-2,6-dione | 3.44 (s, 3H, NCH₃), 4.47 (s, 2H, |
| | | SCH₂C₆H₄Br), 7.30 (t, J = 7.8 Hz, 1H, |
| | | aromatic), 7.43 (t, J = 8.2 Hz, 2H, aromatic), |
| | | 7.68 (s, 1H, aromatic), 13.60 (s (broad), 1H, |
| | | NH). |

### 1.3 Synthesis of ethyl 2-[(7-benzyl-1,3-dimethyl-2,6-dioxo-2,3,6,7-tetrahydro-1H-purin-8-yl)thio]butanoate 1

A suspension of ethyl 2-[(1,3-dimethyl-2,6-dioxo-2,3,6,7-tetrahydro-1H-purin-8-yl)thio]butanoate 6a (1.5 mmol), potassium carbonate (1.5 mmol) and 1-bromo-4-(bromomethyl)benzene 7a (1.5 mmol) in DMF (4 ml) is stirred at room temperature for 3 hours (monitoring by TLC). At the end of the reaction, water is added (20 ml) and the mixture is extracted with toluene (3×10 ml). The combined organic layers are washed with water (5 ml), dried over magnesium sulfate and concentrated. Purification is achieved by chromatography on silica gel (eluent: petroleum ether/acetone) to afford ethyl 2-[(7-benzyl-1,3-dimethyl-2,6-dioxo-2,3,6,7-tetrahydro-1H-purin-8-yl)thio]butanoate 1 as an oil.

Yield: 96%.

MS (ES⁺): 417 (MH⁺, 100).

¹H NMR (CDCl₃): 1.06 (t, J=7.4 Hz, 3H, SCHCH₂CH₃), 1.25 (t, J=7.2 Hz, 3H, OCH₂CH₃), 2.02 (m, 2H, SCHCH₂CH₃), 3.39 (s, 3H, NCH₃), 3.54 (s, 3H, NCH₃), 4.19 (q, J=7.2 Hz, 2H, OCH₂CH₃), 4.41 (t, J=6.9 Hz, 1H, SCHCH₂CH₃), 5.49 (m, 2H, NCH₂C₆H₅), 7.20-7.40 (m, 5H, NCH₂C₆H₅).

Alternatively, compounds may be purified by stirring in diethylether (8 ml/g) and filtration.

### EXAMPLE 2

### Synthesis of ethyl 2-{[7-(3-aminobenzyl)-3-methyl-2,6-dioxo-2,3,6,7-tetrahydro-1H-purin-8-yl]thio}butanoate 14

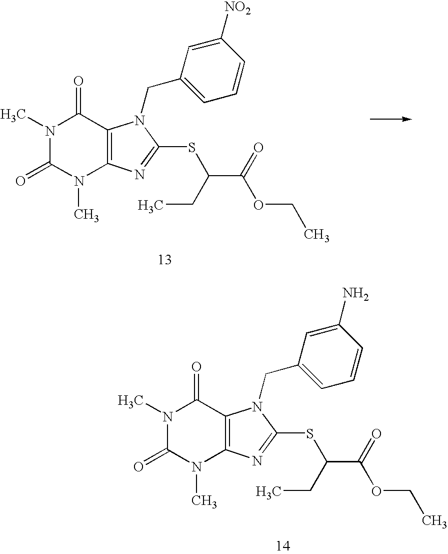

Sodium dithionite (3.36 mmol) is added portionwise (45 min) to a suspension of ethyl 2-{[3-methyl-7-(3-nitrobenzyl)-2,6-dioxo-2,3,6,7-tetrahydro-1H-purin-8-yl]thio}butanoate 13 (1.12 mmol) in a 1:1 mixture of DMF and water (10 ml). The mixture is stirred at room temperature for 2 h30. At the end of the reaction, water (20 ml) and HCl 37% (1 ml) are added and the solution is stirred at room temperature for 16 hours. After basification with ammonia, the mixture is extracted with toluene (3×15 ml). The combined organic layers are washed with water (10 ml), dried over magnesium sulfate and concentrated. Purification is achieved by stirring in diethylether (4 ml) for 4 hours, filtration and drying under vacuum at room temperature for 16 hours and affords ethyl 2-{[7-(3-aminobenzyl)-3-methyl-2,6-dioxo-2,3,6,7-tetrahydro-1H-purin-8-yl]thio}butanoate 14.

Yield: 37%.

mp: 107° C.

MS (ES⁺): 418 (MH⁺).

¹H NMR (CDCl₃): 1.07 (t, J=7.4 Hz, 3H, CHCH₂CH₃), 1.26 (t, J=7.1 Hz, 3H, OCH₂CH₃), 2.05 (m, 2H, CHCH₂CH₃), 3.49 (s, 3H, N³CH₃), 3.8 (m, 2H, NH₂), 4.21 (q, J=7.1 Hz, 2H, OCH₂CH₃), 4.42 (t, J=6.9 Hz, 1H, SCH), 5.32 (m, 2H, N⁷CH₂), 6.57 (d, J=9.3 Hz, 1H, H^{4′}), 6.76 (d, J=9.3 Hz, 1H, H^{6′}), 6.83 (s, 1H, H^{2′}), 7.08 (t, J=9.3 Hz, 1H, H^{5′}), 9.47 (s (broad), 1H, N¹H).

### EXAMPLE 3

### Synthesis of ethyl 2-{[7-(3-bromobenzyl)-1-(2-methoxyethyl)-3-methyl-2,6-dioxo-2,3,6,7-tetrahydro-1H-purin-8-yl]thio}butanoate 3

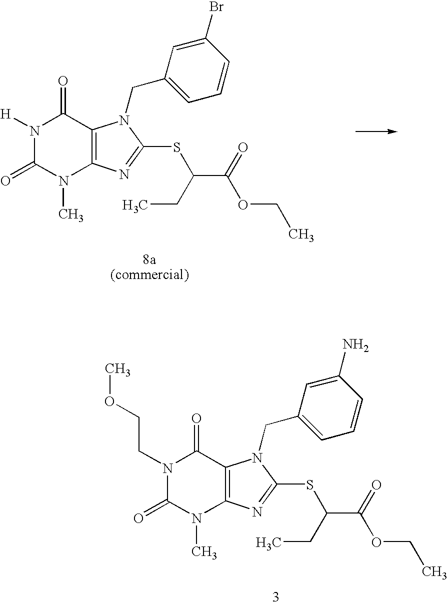

A mixture of ethyl 2-{[7-(3-bromobenzyl)-3-methyl-2,6-dioxo-2,3,6,7-tetrahydro-1H-purin-8-yl]thio}butanoate 8a (commercial) (1.5 mmol), potassium carbonate (1.65 mmol) and 1-bromo-2-methoxyethane (3.0 mmol) in DMF (6 ml) is stirred at room temperature for 48 hours (monitoring by TLC). At the end of the reaction, water (20 ml) is added and the mixture is extracted with toluene (3×10 ml). The combined organic layers are washed with water (5 ml), dried over magnesium sulfate and concentrated. The residue is purified by chromatography on silica gel (eluent: petroleum ether/acetone 95/5) to afford ethyl 2-{[7-(3-bromobenzyl)-1-(2-methoxyethyl)-3-methyl-2,6-dioxo-2,3,6,7-tetrahydro-1H-purin-8-yl]thio}butanoate 3.

Yield: 56%.

mp: 58° C.

MS (ES⁺): 539/541 (MH⁺, 100).

¹H NMR (CDCl₃): 1.06 (t, J=7.4 Hz, 3H, CHCH₂CH₃), 1.26 (t, J=7.1 Hz, 3H, OCH₂CH₃), 2.03 (m, 2H, CHCH₂CH₃), 3.36 (s, 3H, OCH₃), 3.53 (s, 3H, N³CH₃), 3.64 (t, J=5.7 Hz, 2H, CH₂OCH₃), 4.16-4.26 (m, 4H, OCH₂CH₃ and N¹CH₂), 4.44 (t, J=6.9 Hz, 1H, SCH), 5.45 (m, 2H, N⁷CH₂), 7.19 (t, J=7.7 Hz, 1H, C^{5′}), 7.31 (d, J=7.7 Hz, 1H, C^{6′}), 7.42 (d, J=7.7 Hz, 1H, C^{4′}), 7.51 (s, 1H, C^{2′}).

### EXAMPLE 4

### Synthesis of 2-[(7-butyl-3-methyl-2,6-dioxo-2,3,6,7-tetrahydro-1H-purin-8-yl)thio]butanamide 28

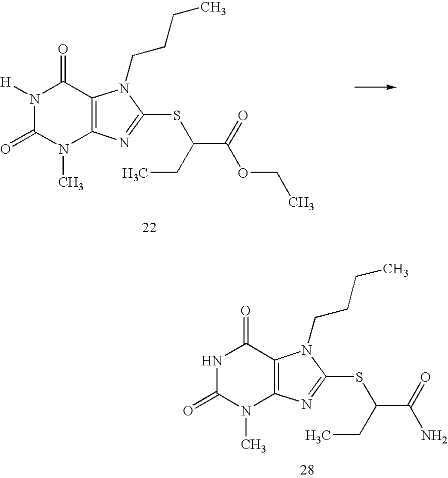

A solution of ethyl 2-[(7-butyl-3-methyl-2,6-dioxo-2,3,6,7-tetrahydro-1H-purin-8-yl)thio]butanoate 22 (5 mmol) in 25 ml of methanol saturated with ammonia is stirred at room temperature for 96 hours. The precipitate is then filtered, washed twice with 2 ml of methanol and dried under vacuum at room temperature for 16 hours to afford 2-[(7-butyl-3-methyl-2,6-dioxo-2,3,6,7-tetrahydro-1H-purin-8-yl)thio]butanamide 28.

Yield: 82%.

mp: 253° C.

MS (ES⁺): 340 (MH⁺).

¹H NMR (d₆-DMSO): 0.92 (t, 3H, CH₂CH₃), 0.98 (t, 3H, CH₂CH₃), 1.30 (m, 2H, N⁷CH₂CH₂CH₂CH₃), 1.70 (m, 2H, N⁷CH₂CH₂), 1.93 (m, 2H, SCH(CONH₂)CH₂CH₃), 3.38 (s, 3H, N³CH₃), 4.20 (t, J=7.2 Hz, 2H, N⁷CH₂), 4.33 (t, J=6.9 Hz, 1H, SCH), 7.32 (s (broad), 1H, NH₂), 7.77 (s (broad), 1H, NH₂), 11.11 (s (broad), 1H, N¹H).

Table I indicates the stereochemical information in the columns headed “configuration”: rac refers to a racemate, “2” consists in the stereochemical assignment for the recognised center according to the IUPAC numbering used in the “IUPAC name” column. Table I indicates also the IUPAC name of the compound, the ion peak observed in mass spectroscopy (MH⁺ or (M⁺.)) and the melting point.

| n° | Configuration | | IUPAC Name | MH⁺(M^{+.}) | mp (° C.) |
|---|---|---|---|---|---|
| 1 | 2 | rac | ethyl 2-[(7-benzyl-1,3-dimethyl-2,6-dioxo-2,3,6,7-tetrahydro-1H-purin-8- | 417 | 72 |
| | | | yl)thio]butanoate | | |
| 2 | 2 | rac | ethyl 2-{[7-(3-bromobenzyl)-1-(2-ethoxy-2-oxoethyl)-3-methyl-2,6-dioxo- | 567/569 | oil |
| | | | 2,3,6,7-tetrahydro-1H-purin-8-yl]thio}butanoate | | |
| 3 | 2 | rac | nzyl)-1-(2-methoxyethyl)-3-methyl-2,6-dioxo-2,3,6,7-tetrahydro-1H-purin-8- | 539/541 | 58 |
| 4 | 2 | rac | ethyl 2-{[7-(3-bromobenzyl)-2,6-dioxo-2,3,6,7-tetrahydro-1H-purin-8- | (467/469) | 90.1 |
| | | | yl]thio}butanoate | | |
| 5 | 2 | rac | ethyl 2-{[7-(3-bromobenzyl)-1,3-dimethyl-2,6-dioxo-2,3,6,7-tetrahydro-1H- | 495/497 | 76 |
| | | | purin-8-yl]thio}butanoate | | |
| 6 | 2 | rac | ethyl 2-{[7-(2-bromobenzyl)-1,3-dimethyl-2,6-dioxo-2,3,6,7-tetrahydro-1H- | 495/497 | 101 |
| | | | purin-8-yl]thio}butanoate | | |
| 7 | 2 | rac | ethyl 2-{[7-(3-bromobenzyl)-1-(cyanomethyl)-3-methyl-2,6-dioxo-2,3,6,7- | 520/522 | 84 |
| | | | tetrahydro-1H-purin-8-yl]thio}butanoate | | |
| 8 | 2 | rac | ethyl 2-{[7-(3-bromobenzyl)-3-methyl-2,6-dioxo-1-propyl-2,3,6,7- | 523/525 | oil |
| | | | tetrahydro-1H-purin-8-yl]thio}butanoate | | |
| 9 | 2 | rac | ethyl 2-{[7-(3-bromobenzyl)-3-methyl-2,6-dioxo-1-(2-oxopropyl)-2,3,6,7- | 537/539 | oil |
| | | | tetrahydro-1H-purin-8-yl]thio}butanoate | | |
| 10 | 2 | rac | ethyl 2-{[7-(3-bromobenzyl)-1-(3-hydroxypropyl)-3-methyl-2,6-dioxo- | 539/541 | oil |
| | | | 2,3,6,7-tetrahydro-1H-purin-8-yl]thio}butanoate | | |
| 11 | 2 | rac | ethyl 2-{[7-(3-bromobenzyl)-3-methyl-2,6-dioxo-1-(2-propynyl)-2,3,6,7- | 519/521 | 97 |
| | | | tetrahydro-1H-purin-8-yl]thio}butanoate | | |
| 12 | 2 | rac | ethyl 2-{[7-(3-methoxybenzyl)-3-methyl-2,6-dioxo-2,3,6,7-tetrahydro-1H- | 433 | 115 |
| | | | purin-8-yl]thio}butanoate | | |
| 13 | 2 | rac | ethyl 2-{[3-methyl-7-(3-nitrobenzyl)-2,6-dioxo-2,3,6,7-tetrahydro-1H-purin- | 448 | 147 |
| | | | 8-yl]thio}butanoate | | |
| 14 | 2 | rac | ethyl 2-{[7-(3-aminobenzyl)-3-methyl-2,6-dioxo-2,3,6,7-tetrahydro-1H- | 418 | 107 |
| | | | purin-8-yl]thio}butanoate | | |
| 15 | 2 | rac | ethyl 2-({7-[4-(aminosulfonyl)benzyl]-3-methyl-2,6-dioxo-2,3,6,7- | 482 | 175.2 |
| | | | tetrahydro-1H-purin-8-yl}thio)butanoate | | |
| 16 | 2 | rac | ethyl 2-{[7-(4-bromobenzyl)-1,3-dimethyl-2,6-dioxo-2,3,6,7-tetrahydro-1H- | 495/497 | oil |
| | | | purin-8-yl]thio}butanoate | | |
| 17 | 2 | rac | ethyl 2-{[7-(cyclohexylmethyl)-1,3-dimethyl-2,6-dioxo-2,3,6,7-tetrahydro- | 423 | oil |
| | | | 1H-purin-8-yl]thio}butanoate | | |
| 18 | 2 | rac | ethyl 2-{[1,3-dimethyl-2,6-dioxo-7-(1-phenylethyl)-2,3,6,7-tetrahydro-1H- | 431 | oil |
| | | | purin-8-yl]thio}butanoate | | |
| 19 | 2 | rac | ethyl 2-{[1,3-dimethyl-2,6-dioxo-7-(2-phenylethyl)-2,3,6,7-tetrahydro-1H- | 431 | oil |
| | | | purin-8-yl]thio}butanoate | | |
| 20 | 2 | rac | ethyl 2-({7-[(3,5-dimethylisoxazol-4-yl)methyl]-3-methyl-2,6-dioxo-2,3,6,7- | 422 | 208 |
| | | | tetrahydro-1H-purin-8-yl}thio)butanoate | | |
| 21 | 2 | rac | ethyl 2-({3-methyl-7-[(5-nitro-2-furyl)methyl]-2,6-dioxo-2,3,6,7-tetrahydro- | 438 | 172 |
| | | | 1H-purin-8-yl}thio)butanoate | | |
| 22 | 2 | rac | ethyl 2-[(7-butyl-3-methyl-2,6-dioxo-2,3,6,7-tetrahydro-1H-purin-8- | 369 | 104 |
| | | | yl)thio]butanoate | | |
| 23 | 2 | rac | ethyl 2-{[7-(3-bromobenzyl)-2,6-dioxo-2,3,6,7-tetrahydro-1H-purin-8- | 411 | 107.9 |
| | | | yl]thio}butanoate | | |
| 24 | 2 | rac | ethyl 2-[(1,7-dihexyl-3-methyl-2,6-dioxo-2,3,6,7-tetrahydro-1H-purin-8- | 481 | oil |
| | | | yl)thio]butanoate | | |
| 25 | 2 | rac | ethyl 2-[(7-hexyl-3-methyl-2,6-dioxo-2,3,6,7-tetrahydro-1H-purin-8- | 397 | 87 |
| | | | yl)thio]butanoate | | |
| 26 | 2 | rac | ethyl 2-[(3-methyl-2,6-dioxo-1,7-dipentyl-2,3,6,7-tetrahydro-1H-purin-8- | 453 | oil |
| | | | yl)thio]butanoate | | |
| 27 | 2 | rac | 2-{[7-(3-bromobenzyl)-3-methyl-2,6-dioxo-2,3,6,7-tetrahydro-1H-purin-8- | 452/454 | 244.15 |
| | | | yl]thio}butanamide | | |
| 28 | 2 | rac | 2-[(7-butyl-3-methyl-2,6-dioxo-2,3,6,7-tetrahydro-1H-purin-8- | 340 | 253 |
| | | | yl)thio]butanamide | | |
| 29 | | achiral | 7-(3-bromobenzyl)-8-[(1-ethylpropyl)thio]-3-methyl-3,7-dihydro-1H-purine- | 437/439 | 167 |
| | | | 2,6-dione | | |
| 30 | 2 | rac | ethyl 2-{8-[(3-bromobenzyl)thio]-1,3-dimethyl-2,6-dioxo-1,2,3,6-tetrahydro- | 495/497 | oil |
| | | | 7H-purin-7-yl}butanoate | | |
| 31 | 2 | rac | ethyl 2-[(7-isobutyl-3-methyl-2,6-dioxo-2,3,6,7-tetrahydro-1H-purin-8- | 369 | 132 |
| | | | yl)thio]butanoate | | |

### EXAMPLE 5

### LBS Binding Assay

[LBS stands for Levetiracetam Binding Site cf. M. Noyer et al., Eur. J. Pharmacol. (1995), 286, 137-146.]

The inhibition constant (Kᵢ) of a compound is determined in competitive binding experiments by measuring the binding of a single concentration of a radioactive ligand at equilibrium with various concentrations of the unlabeled test substance. The concentration of the test substance inhibiting 50% of the specific binding of the radioligand is called the IC₅₀. The equilibrium dissociation constant Kᵢ is proportional to the IC₅₀ and is calculated using the equation of Cheng and Prusoff (Cheng Y. et al., Biochem. Pharmacol. (1972), 22, 3099-3108).

The concentration range usually encompasses 6 log units with variable steps (0.3 to 0.5 log). Assays are performed in mono- or duplicate, each Kᵢ determination is performed on two different samples of test substance.

Cerebral cortex from 200-250 g male Sprague-Dawley rats are homogenised using a Potter S homogeniser (10 strokes at 1,000 rpm; Braun, Germany) in 20 mmol/l Tris-HCl (pH 7.4), 250 mmol/l sucrose (buffer A); all operations are performed at 4° C. The homogenate is centrifuged at 30,000 g for 15 min. The crude membrane pellet obtained is resuspended in 50 mmol/l Tris-HCl (pH 7.4), (buffer B) and incubated 15 min at 37° C., centrifuged at 30,000 g for 15 min and washed twice with the same buffer. The final pellet is resuspended in buffer A at a protein concentration ranging from 15 to 25 mg/ml and stored in liquid nitrogen.

Membranes (150-200 μg of protein/assay) are incubated at 4° C. for 120 min in 0.5 ml of a 50 mmol/l Tris-HCl buffer (pH 7.4) containing 2 mmol/l MgCl₂, 1 to 2 10⁻⁹ mol/l of [³H]-2-[4-(3-azidophenyl)-2-oxo-1-pyrrolidinyl]butanamide and increasing concentrations of the test substance. The non specific binding (NSB) is defined as the residual binding observed in the presence of a concentration of reference substance (e.g. 10⁻³ mol/l levetiracetam) that binds essentially all the receptors. Membrane-bound and free radioligands are separated by rapid filtration through glass fiber filters (equivalent to Whatman GF/C or GF/B; VEL, Belgium) pre-soaked in 0.1% polyethyleneimine and 10⁻³ mol/l levetiracetam to reduce non specific binding. Samples and filters are rinsed by at least 6 ml of 50 mmol/l Tris-HCl (pH 7.4) buffer. The entire filtration procedure does not exceed 10 seconds per sample. The radioactivity trapped onto the filters is counted by liquid scintillation in β-counter (Tri-Carb 1900 or TopCount 9206, Camberra Packard, Belgium, or any other equivalent counter). Data analysis is performed by a computerized non linear curve fitting method using a set of equations describing several binding models assuming populations of independent non-interacting receptors, which obey the law of mass.

Compounds synthesized according to the procedure described in examples 1 to 4 and described in table I are tested in the SV2 binding assay according to the procedure described above, and are found active.

1. A Compound of formula I or an enantiomer, diastereoisomer or mixture thereof (including all possible mixtures of stereoisomers), or a pharmaceutically acceptable salt thereof, 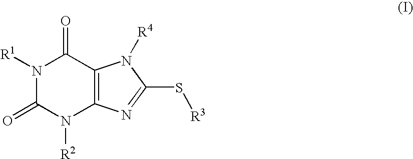
wherein
R¹ is hydrogen or C₁₋₆ alkyl;
R² is hydrogen or C₁₋₄ alkyl;
R³ is a group of formula —CHR⁵R⁶ or a benzyl group;
R⁴ is C₁₋₈ alkyl optionally substituted by alkoxycarbonyl, C₃₋₆ cycloalkyl, aryl or heterocycle;
R⁵ is C₂₋₄ alkyl;
R⁶ is C₂₋₄ alkyl, amido or —COOR⁷;
R⁷ is C₁₋₄ alkyl;
with the proviso that when R¹ is hydrogen, R² is methyl, R³ is —CHR⁵R⁶, R⁶ is ethoxycarbonyl and R⁵ is ethyl, then R⁴ is not methyl, n-propyl, i-propyl, n-pentyl, n-heptyl, 3-bromobenzyl, 4-chlorobenzyl, 4-methylbenzyl or 2-phenylethyl; with the further proviso that when R¹ is hydrogen, R² is methyl, R³ is benzyl, then R⁴ is not i-propyl, n-butyl, 3-methylbutyl, benzyl, phenylethyl or 3-phenylpropyl;
with the further proviso that when R¹ and R² are methyl, R³ is benzyl, R⁴ is not methyl, 3-methylbutyl, benzyl, 3-phenylpropyl or 4-chloro-phenylmethyl; and
with the final proviso that 8-(2-chloro-benzylsulfanyl)-3-methyl-7-octyl-3,7-dihydro-purine-2,6-dione is excluded.

2. The compound according to claim 1, wherein R³ is a benzyl group, and R⁴ is C₁₋₈ alkyl optionally substituted by alkoxycarbonyl.

3. The compound according to claim 1, wherein R³ is a group of formula —CHR⁵R⁶, and R⁴ is C₁₋₈ alkyl optionally substituted by C₃₋₆ cycloalkyl, aryl or heterocycle.

4. The compound according to claim 1, wherein R¹ is hydrogen, methyl, cyanomethyl, 2-ethoxy-2-oxoethyl, 2-methoxyethyl, n-propyl, 2-oxopropyl, 3-hydroxypropyl, 2-propynyl, n-pentyl or n-hexyl.

5. The compound according to claim 1, wherein R² is hydrogen, methyl or n-butyl.

6. The compound according to claim 1, wherein R³ is 3-pentyl, 1-(aminocarbonyl)-propyl, 1-(ethoxycarbonyl)propyl or 3-bromobenzyl.

7. The compound according to claim 1, wherein R⁴ is n-butyl, i-butyl, n-pentyl, n-hexyl, cyclohexylmethyl, benzyl, 2-bromobenzyl, 3-bromobenzyl, 4-bromobenzyl, 3-methoxybenzyl, 3-nitrobenzyl, 3-aminobenzyl, 4-(aminosulfonyl)benzyl, 1-phenylethyl, 2-phenylethyl, (3,5-dimethylisoxazol-4-yl)methyl, (5-nitro-2-furyl)methyl or 1-(ethoxycarbonyl)propyl.

8. The compound according to claim 1, wherein R⁵ is ethyl.

9. The compound according to claim 1, wherein R⁶ is ethyl, amido or ethoxycarbonyl.

10. The compound according to claim 1, wherein R⁷ is ethyl.

11. The compound according to claim 1, wherein R¹ is hydrogen; R² is methyl; R³ is 1-(ethoxycarbonyl)propyl; and R⁴ is 3-methoxybenzyl, 3-nitrobenzyl or (5-nitro-2-furyl)methyl.

12. The compound according to claim 1, wherein the compound is
ethyl 2-[(7-benzyl-1,3-dimethyl-2,6-dioxo-2,3,6,7-tetrahydro-1H-purin-8-yl)thio]butanoate;
ethyl 2-{[7-(3-bromobenzyl)-1-(2-ethoxy-2-oxoethyl)-3-methyl-2,6-dioxo-2,3,6,7-tetrahydro-1H-purin-8-yl]thio}butanoate;
ethyl 2-{[7-(3-bromobenzyl)-1-(2-methoxyethyl)-3-methyl-2,6-dioxo-2,3,6,7-tetrahydro-1H-purin-8-yl]thio}butanoate;
ethyl 2-{[7-(3-bromobenzyl)-2,6-dioxo-2,3,6,7-tetrahydro-1H-purin-8-yl]thio}butanoate;
ethyl 2-{[7-(3-bromobenzyl)-1,3-dimethyl-2,6-dioxo-2,3,6,7-tetrahydro-1H-purin-8-yl]thio}butanoate;
ethyl 2-{[7-(2-bromobenzyl)-1,3-dimethyl-2,6-dioxo-2,3,6,7-tetrahydro-1H-purin-8-yl]thio}butanoate;
ethyl 2-{[7-(3-bromobenzyl)-1-(cyanomethyl)-3-methyl-2,6-dioxo-2,3,6,7-tetrahydro-1H-purin-8-yl]thio}butanoate;
ethyl 2-{[7-(3-bromobenzyl)-3-methyl-2,6-dioxo-1-propyl-2,3,6,7-tetrahydro-1H-purin-8-yl]thio}butanoate;
ethyl 2-{[7-(3-bromobenzyl)-3-methyl-2,6-dioxo-1-(2-oxopropyl)-2,3,6,7-tetrahydro-1H-purin-8-yl]thio}butanoate;
ethyl 2-{[7-(3-bromobenzyl)-1-(3-hydroxypropyl)-3-methyl-2,6-dioxo-2,3,6,7-tetrahydro-1H-purin-8-yl]thio}butanoate;
ethyl 2-{[7-(3-bromobenzyl)-3-methyl-2,6-dioxo-1-(2-propynyl)-2,3,6,7-tetrahydro-1H-purin-8-yl]thio}butanoate;
ethyl 2-{[7-(3-methoxybenzyl)-3-methyl-2,6-dioxo-2,3,6,7-tetrahydro-1H-purin-8-yl]thio}butanoate;
ethyl 2-{[3-methyl-7-(3-nitrobenzyl)-2,6-dioxo-2,3,6,7-tetrahydro-1H-purin-8-yl]thio}butanoate;
ethyl 2-{[7-(3-aminobenzyl)-3-methyl-2,6-dioxo-2,3,6,7-tetrahydro-1H-purin-8-yl]thio}butanoate;
ethyl 2-({7-[4-(aminosulfonyl)benzyl]-3-methyl-2,6-dioxo-2,3,6,7-tetrahydro-1H-purin-8-yl}thio)butanoate;
ethyl 2-{[7-(4-bromobenzyl)-1,3-dimethyl-2,6-dioxo-2,3,6,7-tetrahydro-1H-purin-8-yl]thio}butanoate;
ethyl 2-{[7-(cyclohexylmethyl)-1,3-dimethyl-2,6-dioxo-2,3,6,7-tetrahydro-1H-purin-8-yl]thio}butanoate;
ethyl 2-{[1,3-dimethyl-2,6-dioxo-7-(1-phenylethyl)-2,3,6,7-tetrahydro-1H-purin-8-yl]thio}butanoate;
ethyl 2-{[1,3-dimethyl-2,6-dioxo-7-(2-phenylethyl)-2,3,6,7-tetrahydro-1H-purin-8-yl]thio}butanoate;
ethyl 2-({7-[(3,5-dimethylisoxazol-4-yl)methyl]-3-methyl-2,6-dioxo-2,3,6,7-tetrahydro-1H-purin-8-yl}thio)butanoate;
ethyl 2-({3-methyl-7-[(5-nitro-2-furyl)methyl]-2,6-dioxo-2,3,6,7-tetrahydro-1H-purin-8-yl}thio)butanoate;
ethyl 2-[(7-butyl-3-methyl-2,6-dioxo-2,3,6,7-tetrahydro-1H-purin-8-yl)thio]butanoate;
ethyl 2-{[7-(3-bromobenzyl)-2,6-dioxo-2,3,6,7-tetrahydro-1H-purin-8-yl]thio}butanoate;
ethyl 2-[(1,7-dihexyl-3-methyl-2,6-dioxo-2,3,6,7-tetrahydro-1H-purin-8-yl)thio]butanoate;
ethyl 2-[(7-hexyl-3-methyl-2,6-dioxo-2,3,6,7-tetrahydro-1H-purin-8-yl)thio]butanoate;
ethyl 2-[(3-methyl-2,6-dioxo-1,7-dipentyl-2,3,6,7-tetrahydro-1H-purin-8-yl)thio]butanoate;
2-{[7-(3-bromobenzyl)-3-methyl-2,6-dioxo-2,3,6,7-tetrahydro-1H-purin-8-yl]thio}butanamide;
2-[(7-butyl-3-methyl-2,6-dioxo-2,3,6,7-tetrahydro-1H-purin-8-yl)thio]butanamide;
7-(3-bromobenzyl)-8-[(1-ethylpropyl)thio]-3-methyl-3,7-dihydro-1H-purine-2,6-dione;
ethyl 2-{8-[(3-bromobenzyl)thio]-1,3-dimethyl-2,6-dioxo-1,2,3,6-tetrahydro-7H-purin-7-yl}butanoate; or
and ethyl 2-[(7-isobutyl-3-methyl-2,6-dioxo-2,3,6,7-tetrahydro-1H-purin-8-yl)thio]butanoate.

13. The compound according to claim 1, wherein the compound is
ethyl 2-{[7-(3-methoxybenzyl)-3-methyl-2,6-dioxo-2,3,6,7-tetrahydro-1H-purin-8-yl]thio}butanoate;
ethyl 2-{[3-methyl-7-(3-nitrobenzyl)-2,6-dioxo-2,3,6,7-tetrahydro-1H-purin-8-yl]thio}butanoate; or
and ethyl 2-({3-methyl-7-[(5-nitro-2-furyl)methyl]-2,6-dioxo-2,3,6,7-tetrahydro-1H-purin-8-yl}thio)butanoate.

14. A method of treating an epileptic disorder, epileptogenesis, seizure disorders, Parkinson's disease, dyskinesia, incontinence, or neuropathic pain, the method comprising administering to a patient in need thereof a therapeutically effective amount of a compound according to formula II 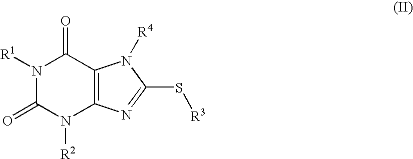
or an enantiomer, diastereoisomer, or mixture thereof (including all possible mixtures of stereoisomers), or a pharmaceutically acceptable salt thereof,
wherein
R¹ is hydrogen or C₁₋₆ alkyl;
R² is hydrogen or C₁₋₄ alkyl;
R³ is a group of formula —CHR⁵R⁶ or a benzyl group;
R⁴ is C₁₋₁₈ alkyl optionally substituted by alkoxycarbonyl, C₃₋₆ cycloalkyl, aryl or heterocycle;
R⁵ is hydrogen or C₁₋₄ alkyl;
R⁶ is C₁₋₄ alkyl, amido or —COOR⁷; and
R⁷ is C₁₋₄ alkyl.

15. The method according to claim 14, wherein R² is methyl, R³ is a group of formula —CHR⁵R⁶ with R⁵ being C₂₋₄ alkyl, R⁶ being amido or —COOR⁷ and R⁷ being methyl or ethyl.

16. The method according to claim 15, wherein the compound is
ethyl 2-[(7-heptyl-3-methyl-2,6-dioxo-2,3,6,7-tetrahydro-1H-purin-8-yl)thio]butanoate;
7-(3-bromobenzyl)-3-methyl-8-(propylthio)-3,7-dihydro-1H-purine-2,6-dione;
ethyl 2-[(3-methyl-2,6-dioxo-7-pentyl-2,3,6,7-tetrahydro-1H-purin-8-yl)thio]butanoate;
ethyl 2-{[7-(3-bromobenzyl)-3-methyl-2,6-dioxo-2,3,6,7-tetrahydro-1H-purin-8-yl]thio}butanoate;
ethyl 2-[(3-methyl-2,6-dioxo-7-propyl-2,3,6,7-tetrahydro-1H-purin-8-yl)thio]butanoate;
7-(3-bromobenzyl)-8-[(3-chloro-2-hydroxypropyl)thio]-3-methyl-3,7-dihydro-1H-purine-2,6-dione; or
and ethyl 2-{[7-(3-bromobenzyl)-3-methyl-2,6-dioxo-2,3,6,7-tetrahydro-1H-purin-8-yl]thio}propanoate.

17. The method according to claim 14 wherein the disease is an epileptic disorder.

18. A compound of formula III 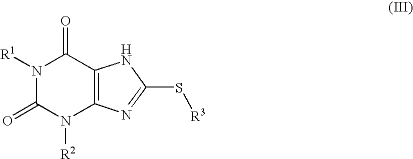
wherein
R¹ is hydrogen or C₁₋₆ alkyl;
R² is hydrogen or C₁₋₄ alkyl;
R³ is a group of formula —CHR⁵R⁶ or a benzyl group;
R⁵ is C₂₋₄ alkyl;
R⁶ is C₂₋₄ alkyl, amido or —COOR⁷; and
R⁷ is C₁₋₄alkyl;

19. A compound that is
ethyl 2-[(1,3-dimethyl-2,6-dioxo-2,3,6,7-tetrahydro-1H-purin-8-yl)thio]butanoate;
ethyl 2-[(2,6-dioxo-2,3,6,7-tetrahydro-1H-purin-8-yl)thio]butanoate;
ethyl 2-[(3-methyl-2,6-dioxo-2,3,6,7-tetrahydro-1H-purin-8-yl)thio]butanoate;
8-[(1-ethylpropyl)thio]-3-methyl-3,7-dihydro-1H-purine-2,6-dione; or
8-[(3-bromobenzyl)thio]-3-methyl-3,7-dihydro-1H-purine-2,6-dione.
